(19) United States
(12) Patent Application Publication
(10) Pub. No.: US 2009/0324489 A1
(43) Pub. Date: Dec. 31, 2009

(54) Wortmannin conjugates and uses thereof

(76) Inventors: Hushan Yuan, West Roxbury, MA (US); Ji Luo, Cambridge, MA (US); Ralph Weissleder, Peabody, MA (US); Lewis Cantley, Cambridge, MA (US); Lee Josephson, Reading, MA (US)

Correspondence Address: CLARK & ELBING LLP, 101 FEDERAL STREET, BOSTON, MA, 02110, US

(21) Appl. No.: 11/991,111

(22) PCT Filed: Aug. 31, 2006
(86) PCT No.: PCT/US06/34046

§ 371 Date: Aug. 11, 2009

Related U.S. Application Data
(60) Provisional application No. 60/713,242, filed on Sep. 1, 2005.

(51) Int. Cl. A61K 47/48; A61K 31/366; A61K 51/00; A61K 39/395; A61K 31/58; A61K 31/566; C07J 71/00; C07D 311/78; C07J 1/00

(52) U.S. Cl. 424/1.11; 424/178.1; 514/2; 514/44 R; 514/172; 514/179; 514/453; 530/300; 530/391.7; 536/123.1; 540/78; 549/276; 552/613

(57) ABSTRACT

The invention features conjugates of wortmannin, and wortmannin derivatives, and their use as inhibitors of PI3-kinase activity in treating cancer, inflammatory diseases, and C. albicans infections.

### STATEMENT AS TO FEDERALLY SPONSORED RESEARCH

This invention was sponsored in part by Grant Nos. CA85240, HL078641, EB004626, GM41890, and CA89021 from the National Institutes of Health. The Government has certain rights to this invention.

### BACKGROUND OF THE INVENTION

The invention relates to conjugates of wortmannin, and wortmannin derivatives, and their use in treating disease.

Wortmannin is a steroid-like molecule that was originally isolated from the soil bacteria Pennicillium wortmannii. The biosynthetic production of wortmannin is well known in the art. See for example U.S. Pat. No. 6,703,414, incorporated herein by reference, which describes the typical fermentation, extraction, and purification process of producing wortmannin.

The structure of wortmannin is shown in structure I below:

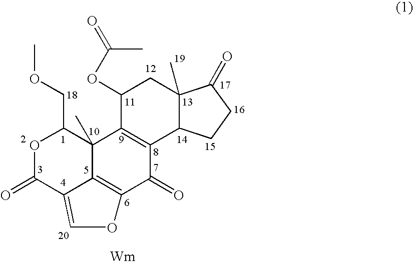

Wortmannin has been shown to react covalently with a lysine in the ATP binding site of the catalytic subunit of phosphoinositol-3-OH kinase (PI3K) (Wymann et al., Mol. Cell Biol. 16:1722 (1996)). P13K plays a central role in cell proliferation and immune regulation, which suggests that PI3K inhibitors might be developed as anti-cancer or anti-inflammatory drugs (Wetzker et al., Current Pharmaceutical Design 10:1915(2004); Luo et al., 4:257 (2003); Wymann et al., Trends Pharmacol. Sci. 24:366 (2003)). PI3K activity is involved the action of hormones including insulin (Ruderman et al., Proc. Natl. Acad. Sci. USA 87:1411 (1990); Nakanishi, T., Proc. Natl. Acad. Sci. USA 92:5317 (1995)) and growth factors (PDGF, VEGF Her2/neu, EGF) (Fresno Vara et al., Cancer Treat Rev. 30:193 (2004)). Though wortmannin is a high affinity inhibitor of PI3K, with considerable specificity for this enzyme, wortmannin inhibits other kinases including mTOR, DNA-PK and a polo-like kinase (Wipf et al., Org. Biomol. Chem. 3:2055 (2005)).

Wortmannin and wortmannin-like compounds have long been investigated as potential anti-inflammatory agents (1970's-present) (Wiesinger et al., Experientia 30:135 (1974); Gunther et al., Immunopharmacol. Immunotoxicol. 11:559 (1989); Yano et al., J. Biol Chem. 268:25846 (1993); Wymann et al., Biochem. Soc. Trans. 3 l(Pt 1):275 (2003)) and more recently for their anti-cancer activities (mid-1990's-present) (see, for example, Powis et al., Cancer Metastasis Rev. 13:91 (1994); Schultz et al., Anticancer Res. 15:1135 (1995)). Wortmannin is growth inhibitory for cancers in different animal models (Bondar et al., Mol. Cancer Ther. 1:989 (2002); Boehle et al., Langenbecks Arch. Surg. 387:234 (2002); Lemke et al., Cancer Chemother. Pharmacol. 44:491 (1999); Schultz et al., Anticancer Res. 15:1135 (1995)). Wortmannin has also been investigated as an anti-fungal agent due to its toxicity to the pathogen Candida albicans (Jayasinthe et al., J. Nat. Prod. 69:439(2006)).

Despite a large literature suggesting Wortmannin or Wortmannin-like compounds could be used as pharmaceuticals, two general problems, toxicity and instability, have hampered their development into drugs. Toxicity results from the uncontrolled and broad distribution of wortmannin, and its ability to inhibit of PI3 kinases (or other kinases) in both normal and pathological tissues. The toxicity is presumably related to the essential role played by PI3K in a wide variety of essential cell functions, and the inability to obtain PI3K inhibitors that are specific for pathological rather than normal tissues. Early literature described Wortmannin toxicity as hemorrhagic (Bosch et al., Mycopathologia 108:73 (1989); Mirocha et al., Bioactive Molecules 10:213 (1989)), with more recent works focusing on its hepatic and myelosuppressive toxicities (Ihle et al., Mol. Cancer Ther. 3:763 (2004); Wipf et al., Org. Biomol. Chem. 2:1911 (2004)).

Development of Wortmannin as a pharmaceutical has also hindered by its instability in various biological media, with a half-life of 8-13 minutes in tissue culture media (Holleran et al., Anal. Biochem. 323:19 (2003)). For further comments on wortmannin's instability see Wipf et al., Org. Biomol. Chem. 3:2055 (2005)).

Several researchers have attempted to overcome the deficiencies of wortmannin by modifying the wortmannin molecule. Wortmannin conjugates have been made by Vitarkowski who conjugated Wortmannin at C11 to HPMA (Varticovski et al., Journal of Controlled Release 74:275 (2001)) and by Yu, who conjugated Wortmannin at C17 to PEG (Yu et al., Cancer Biol. Ther. 4:538 (2005)). Creemer examined the properties of Wortmannin derivatives made by modifications of Wortmannin at a variety of positions including C17 and C11 (see Creemer et al., J. Med. Chem. 39:5021 (1996); and U.S. Pat. No. 5,480,906). Norman evaluated Wortmannin modified at different positions, such as C20 and C11 (Norman et al., J. Med. Chem. 39:1106 (1996)), with one study focusing on the modification of the furan ring at C20 with small molecules (Norman et al., Bioorganic & Medicinal Chemistry Letters 5:1183 (1995).

Other efforts to develop Wortmannin based pharmaceuticals have focused on the modification of Wortmannin at the C20 position by amines or thiol compounds ((See Ihle et al., Mol. Cancer Ther. 3:763 (2004); and Wipf et al., Org. Biomol. Chem. 2:1911 (2004)). A compound termed PX-866 is formed by the reaction of Wortmannin with diallylamine at the C20 position. Although the compound showed some limited improved potency, this compound has a limited plasma half-life which may limit its use as a pharmaceutical. Moreover, these authors propose that the improved activity of some wortmannins modified at C20 over Wortmannin is due to either an improved fit into the ATP site of PI3K or because the C20 substituents serve as superior leaving groups, enhancing the rate of attack by the lysine residue and covalent modification of the enzyme. Wortmannin modifications based on this hypothesis are restricted by the need for the modified wortmannin to fit into the active site of PI3K, the presumed molecular target.

There is a need for a broad and versatile wortmannin based chemistry that can yield compounds that overcome the problems with wortmannin, principally instability and toxicity. Additionally, there is a need for new compounds whose predominant mode of action maybe inhibition of PI3K (or similar kinases) in specific cells, particularly acute for the alpha and beta isoforms of PI3K which are ubiquitous and whose enzyme activity is crucial for the cancerous state.

### SUMMARY OF THE INVENTION

We have discovered novel, stable wortmannin derivatives and conjugates that can overcome the deficiencies described above and can be delivered preferentially to pathological cells and tissues. The compounds of the invention are stable and regenerate wortmannin. This latter feature permits the design of wortmannin-based compounds without regard to whether they fit into the active site of a PI3K or other molecular receptors of wortmannin.

In a first aspect, the invention features a compound of formula I:

W−L−T (I)

In formula I, W is a wortmannin C20 derivative; T is a non-naturally occurring targeting group; and L is a linker which forms a covalent bond with the wortmannin C20 derivative at position C20 and forms a covalent bond with the targeting group. The compound of formula I includes a thioether or amine substituent at position C20 of the wortmannin C20 derivative.

In a second aspect, the invention features a substantially pure compound of formula I (above), wherein W is a wortmannin C20 derivative; T is a targeting group; and L is a linker which forms a covalent bond with the wortmannin C20 derivative at position C20 and forms a covalent bond with the targeting group. The compound of formula I includes a thioether or amine substituent at position C20 of the wortmannin C20 derivative.

In a third aspect, the invention features a pharmaceutical composition including a compound of formula I (above) and a pharmaceutically acceptable carrier or diluent, wherein W is a wortmannin C20 derivative; T is a targeting group; and L is a linker which forms a covalent bond with the wortmannin C20 derivative at position C20 and forms a covalent bond with the targeting group. The compound of formula I includes a thioether or amine substituent at position C20 of the wortmannin C20 derivative.

In an embodiment of the above-aspects, the wortmannin C20 derivative is obtained from a wortmannin-like compound selected from viridin, viridiol, demethoxyviridin, demethoxyviridiol, wortmannin, wortmannolone, 17-hydroxywortmannin, 11-desacetoxywortmannin, and Δ9,11 dehydrodesacetoxywortmannin.

In some embodiments of the above-aspects, the linker is further described by formula IIa:

G²-(X¹)—(R₁₀)-(Z²)ₛ-(Y¹)ᵤ-(Z¹)ₒ-G¹ (IIa)

In formula IIa G¹ is a bond between the linker and the targeting group; G² is a bond between the linker and the wortmannin C20 derivative; X¹ is S or NR₁₁; each of Z¹ and Z² is, independently, selected from O, S, and NR₁₂; each of o, s, and u is, independently, 0 or 1; Y¹ is selected from carbonyl, thiocarbonyl, sulphonyl, and phosphoryl; R₁₂ is selected from hydrogen, C₁₋₁₀ alkyl, C₂₋₁₀ alkene, C₂₋₁₀ alkyne, and C₅₋₁₀ arylgroup; R₁₁ is selected from hydrogen, C₁₋₁₀ alkyl, C₂₋₁₀ alkene, C₂₋₁₀ alkyne, and C₅₋₁₀ arylgroup; and R₁₀ is a C₁₋₁₀ alkyl, C₁₋₁₀ heteroalkyl, C₂₋₁₀ alkene, a C₂₋₁₀ alkyne, a C₅₋₁₀ aryl, a cyclic system of 3 to 10 atoms, or —(CH₂CH₂O)_{q}CH₂CH₂ in which q is an integer of 1 to 8; or R₁₀ and R₁₁ combine to form a cyclic system of 3 to 10 atoms. In one particular embodiment T and W are linked using an amino acid, such as proline, lysine, 6-amino-N-methyl hexanoic acid, or 6-amino hexanoic acid, and is described by formula IIb:

G²-(NR₁₃)—R₁₄—C(O)-G¹ (IIb)

In formula IIb G¹ is a bond between the linker and the targeting group; G² is a bond between the linker and the wortmannin C20 derivative; R₁₃ is selected from hydrogen, C₁₋₁₀ alkyl, C₁₋₁₀ heteroalkyl, C₂₋₁₀ alkene, C₂₋₁₀ alkyne, and C₅₋₁₀ arylgroup; and R₁₄ is a C₁₋₁₀ alkyl, C₁₋₁₀ heteroalkyl, C₂₋₁₀ alkene, a C₂₋₁₀ alkyne, a C₅₋₁₀ aryl, a cyclic system of 3 to 10 atoms, or —(CH₂CH₂O)_{q}CH₂CH₂— in which q is an integer of 1 to 8; or R₁₃ and R₁₄ combine to form a cyclic system of 3 to 10 atoms. In still other embodiments the linker is a bond formed by reaction of thiol group or amino group of targeting group T with wortmannin or a wortmannin-like compound, such that linker L is only a bond between wortmannin C20 derivative W and targeting group T.

In another embodiment of the above-aspects, the targeting group is a peptide or peptidomimetic (e.g., bombesin-like peptides, somatostatin-like peptide, RGD peptides, or EPPT1 peptide), low molecular weight ligand (e.g., methotrexate, trimetrexate, or folate), protein (e.g., an antibody or fragment thereof, such as rituximab, cetuximab, trastuzumab, bevacizumab, and abciximab), polymer (e.g., a polymer including a polypeptide, polysaccharide, or polyethyleneglycol), solid support, anticancer agent (e.g., alkylating agents, folic acid antagonists, pyrimidine antagonists, purine antagonists, antimitotic agents, DNA topomerase II inhibitors, DNA topomerase I inhibitors, taxanes, DNA intercalators, aromatase inhibitors, 5-alpha-reductase inhibitors, estrogen inhibitors, androgen inhibitors, gonadotropin releasing hormone agonists, retinoic acid derivatives, or hypoxia selective cytotoxins), or anti-inflammatory agent (e.g., non steroidal anti-inflammatory drugs, COX-2 inhibitors, anti-inflammatory biologics, and corticosteroids). The targeting group can be, without limitation, any agent identified in Tables 5a or 5b.

In any of the above-aspects, the wortmannin conjugate of the invention is further described by any of formulas IIIa to IIIi:

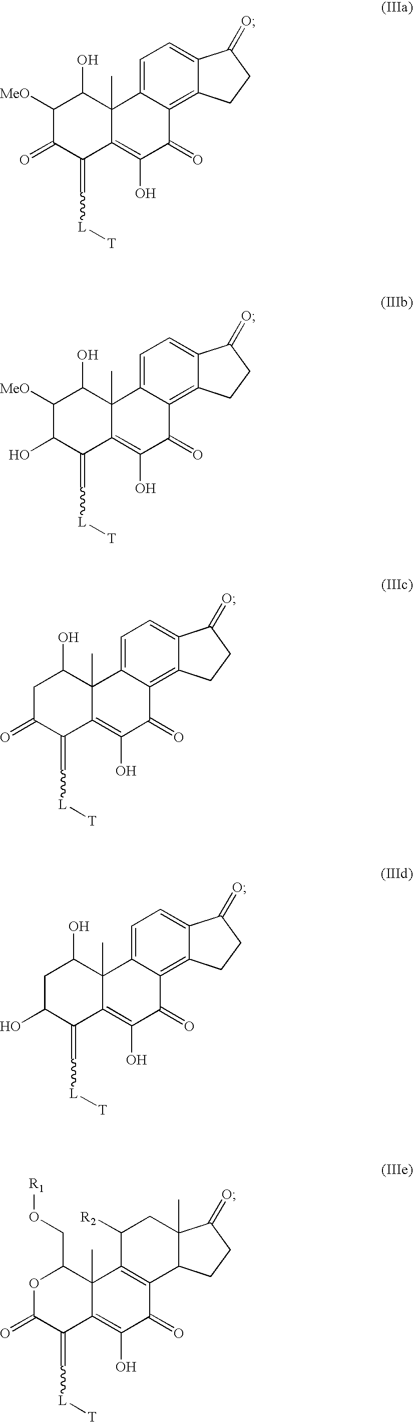

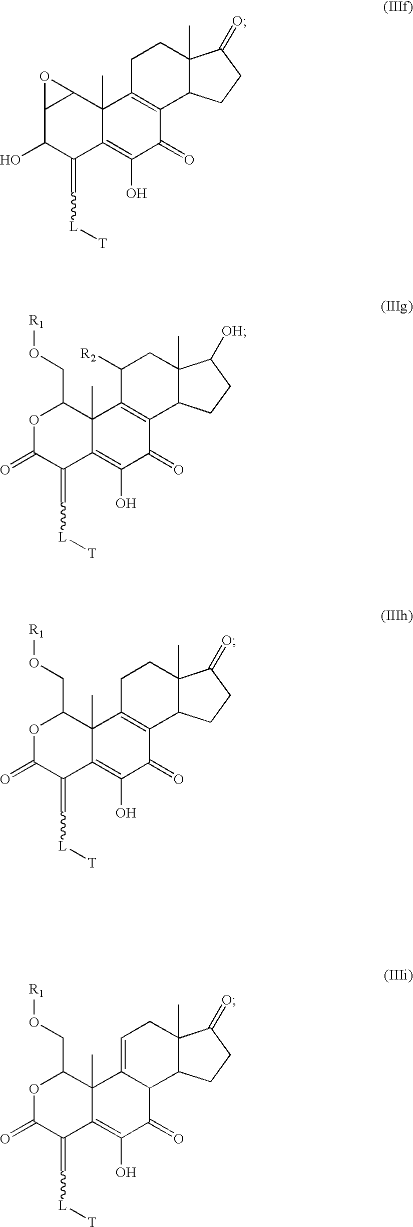

In formulas IIIa to IIIi, T is a targeting group; L is a linker; R₂ is OH, OR₃, or OC(O)R₃; and each of R₁ and R₃ is, independently, selected from C₁₋₁₀ alkyl, C₁₋₁₀ heteroalkyl, C₂₋₁₀ alkene, and C₂₋₁₀ alkyne. In each of formulas IIIa to IIIi, the compound includes a thioether or amine substituent where linker L attaches to position C20 of the wortmannin C20 derivative. Desirably, R₁ and R₃ are methyl.

In one embodiment of the above aspects, L is a bond linking the wortmannin C20 derivative to the targeting group. In such instances, the thioether or amine substituent is formed via a ring-opening attack of the C20 position by a primary amine, secondary amine, or thiol present on the targeting group.

In a fourth aspect, the invention features a pharmaceutical composition including a wortmannin conjugate described herein in any pharmaceutically acceptable form and a pharmaceutically acceptable carrier or diluent.

In a fifth aspect, the invention features an article comprising a compound of formula I (above), wherein W is a wortmannin C20 derivative; T is a solid support on or within said article; L is a linker which forms a covalent bond with the wortmannin C20 derivative at position C20 and forms a covalent bond with the solid support. The composition includes a thioether or amine substituent at position C20 of the wortmannin C20 derivative.

In an embodiment of the above-aspect, the article is an implantable medical device. Desirably, the implantable medical device is a stent or a drug delivery device.

In a sixth aspect, the invention features a method for reducing PI3 kinase activity in a cell by contacting the cell with a wortmannin conjugate of the invention in an amount sufficient to reduce the PI3 kinase activity.

In a seventh aspect, the invention features a method for treating an inflammatory condition in a mammal by administering to the mammal a wortmannin conjugate of the invention in an amount sufficient to treat the inflammatory condition.

In an eighth aspect, the invention features a method for treating a proliferative disorder in a mammal by administering to the mammal a wortmannin conjugate of the invention in an amount sufficient to treat the proliferative disorder.

In a ninth aspect, the invention features a method for treating a Candida albicans infection in a mammal by administering to the mammal a wortmannin conjugate of the invention in an amount sufficient to treat the infection.

In an tenth aspect, the invention features a process for the preparation of a compound of formula IV by reacting a compound of formula V with a targeting group bearing a primary or secondary amine.

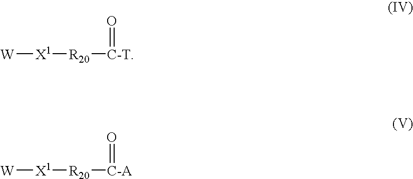

In formulas IV and V, W is a wortmannin C20 derivative; T is a targeting group bearing a primary or secondary amine; A is N-hydroxysuccinimidyl ester or N-hydroxysulfosuccinimidyl ester; X¹ is S or NR₂₁; R₂₁ is selected from hydrogen, C₁₋₁₀ alkyl, C₁₋₁₀ heteroalkyl, C₂₋₁₀ alkene, C₂₋₁₀ alkyne, and C₅₋₁₀ aryl; and R₂₀ is a C₁₋₁₀ alkyl, C₁₋₁₀ heteroalkyl, a C₂₋₁₀ alkene, a C₂₋₁₀ alkyne, a C₅₋₁₀ aryl, a cyclic system of 3 to 10 atoms, or —(CH₂CH₂O)_{q}CH₂CH₂— in which q is an integer of 1 to 8; or R₂₀ and R₂₁ combine to form a cyclic system of 3 to 10 atoms.

In an eleventh aspect, the invention features a process for the preparation of a wortmannin conjugate of the invention by reacting a compound of formula VI with a targeting group bearing a primary amine.

W—(X¹)—R₃₀ (VI)

In formula VI, W is a wortmannin C20 derivative; X¹ is S or NR₃₁; each of R₃₀ and R₃, is, independently, selected from C₁₋₁₀ alkyl, C₂₋₁₀ alkene, C₂₋₁₀ alkyne, C₅₋₁₀ arylgroup, and a cyclic system of 3 to 10 atoms; or R₃₀ and R₃₁ combine to form a cyclic system of 3 to 10 atoms. In certain embodiments, the targeting group is attached to a linker bearing a primary amino group for reaction with the compound of formula VI. In an alternative embodiment the targeting group itself bears a primary amino group for reaction with the compound of formula VI.

In an twelfth aspect, the invention features a compound of formula VII:

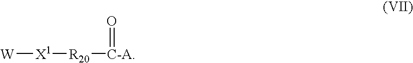

In formula VII, W is a wortmannin C20 derivative; A is N-hydroxysuccinimidyl ester or N-hydroxysulfosuccinimidyl ester; X¹ is S or NR₂₁; R₂₁ is selected from hydrogen, C₁₋₁₀ alkyl, C₁₋₁₀ heteroalkyl, C₂₋₁₀ alkene, C₂₋₁₀ alkyne, and C₅₋₁₀ aryl; and R₂₀ is a C₁₋₁₀ alkyl, C₁₋₁₀ heteroalkyl, a C₂₋₁₀ alkene, a C₂₋₁₀ alkyne, a C₅₋₁₀ aryl, a cyclic system of 3 to 10 atoms, or —(CH₂CH₂O)_{q}CH₂CH₂— in which q is an integer of 1 to 8; or R₂₀ and R₂₁ combine to form a cyclic system of 3 to 10 atoms. Desirably, the compound of formula V is the N-hydroxysuccinimide ester of wortmannin C20-N(Me)-hexanoic acid or the N-hydroxysuccinimide ester of wortmannin C20-NH-hexanoic acid.

In an thirteenth aspect, the invention features a compound of formula VIII or a salt thereof:

W—N(CH₃)—[CH₂]ₙ—COOH (VIII)

In formula VIII, W is a wortmannin C20 derivative; and n is an integer of 2 to 10. In one embodiment, n is 2, 3, 4, 5, 6, 7, 8, 9, or 10.

In the generic descriptions of compounds of this invention, the number of atoms of a particular type in a substituent group is generally given as a range, e.g., an alkyl group containing from 1 to 10 carbon atoms or C₁₋₁₀ alkyl. Reference to such a range is intended to include specific references to groups having each of the integer number of atoms within the specified range. For example, an alkyl group from 1 to 10 carbon atoms includes each of C₁, C₂, C₃, C₄, C₅, C₆, C₇, C₈, C₉, and C₁₀. A C₁₋₁₀ heteroalkyl, for example, includes from 1 to 10 carbon atoms in addition to one or more heteroatoms. Other numbers of atoms and other types of atoms may be indicated in a similar manner.

By “C₁₋₁₀ alkyl” is meant a branched or unbranched saturated hydrocarbon group, having 1 to 10 carbon atoms, inclusive. An alkyl may optionally include monocyclic, bicyclic, or tricyclic rings, in which each ring desirably has three to six members. The alkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₁₀ alkene” is meant a branched or unbranched hydrocarbon group containing one or more double bonds, desirably having from 2 to 10 carbon atoms. A C₂₋₁₀ alkene may optionally include monocyclic, bicyclic, or tricyclic rings, in which each ring desirably has five or six members. The C₂₋₁₀ alkene group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₁₀ alkyne” is meant a branched or unbranched hydrocarbon group containing one or more triple bonds, desirably having from 2 to 10 carbon atoms. A C₂₋₁₀ alkyne may optionally include monocyclic, bicyclic, or tricyclic rings, in which each ring desirably has five or six members. The C₂₋₁₀ alkene group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₁₋₁₀ heteroalkyl” is meant a branched or unbranched alkyl, alkenyl, or alkynyl group having from 1 to 10 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

By “C₅₋₁₀ aryl” or “aryl” is meant an aromatic group having a ring system with conjugated π electrons (e.g., phenyl, or imidazole ). The ring of the aryl group is preferably 5 to 10 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

The term “cyclic system” refers to a compound that contains one or more covalently closed ring structures, in which the atoms forming the backbone of the ring are composed of any combination of the following: carbon, oxygen, nitrogen, sulfur, and phosphorous. The cyclic system may be substituted or unsubstituted. Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is an alkyl group.

By “hydroxyalkyl” is meant a chemical moiety with the formula —(R)—OH, wherein R is an alkyl group.

By “alkoxy” is meant a chemical substituent of the formula —OR, wherein R is an alkyl group.

By “aryloxy” is meant a chemical substituent of the formula —OR, wherein R is a C₅ l₀ aryl group.

By “alkylthio” is meant a chemical substituent of the formula —SR, wherein R is an alkyl group.

By “arylthio” is meant a chemical substituent of the formula —SR, wherein R is a C₅₋₁₀ aryl group.

By “quaternary amino” is meant a chemical substituent of the formula —(R)—NR′)(R″)(R′″)⁺, wherein R, R′, R″, and R′″ are each independently a C₁₋₁₀ alkyl, C₂₋₁₀ alkene, C₂₋₁₀ alkyne, or C₅₋₁₀ aryl. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

For any reference provided herein to a numbered position in a wortmannin conjugate, wortmannin C20 derivative, or wortamannin-like compound, the recited position is defined by the numbering scheme shown below showing the skeletons for the ring-open (conjugates and derivatives) and ring closed (wortmannin-like compounds) structures. In the structures below,

X is C or O and X¹ is a thioether or amine substituent (e.g., —SR or —NRR′), which is covalently tethered to a linker and/or targeting group.

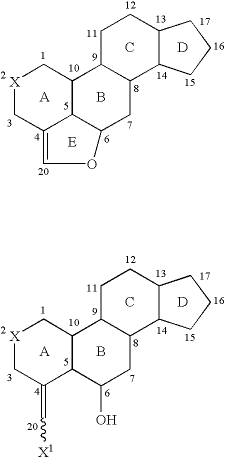

The term “administration” or “administering” refers to a method of giving a dosage of a pharmaceutical composition to a mammal, wherein the composition of the invention is administered by a route selected from, without limitation, inhalation, ocular administration, nasal instillation, parenteral administration, dermal administration, transdermal administration, buccal administration, rectal administration, sublingual administration, perilingual administration, nasal administration, topical administration and oral administration. Parenteral administration includes intravenous, intraperitoneal, subcutaneous, and intramuscular administration. The preferred method of administration can vary depending on various factors, e.g., the components of the pharmaceutical composition, site of the potential or actual disease and severity of disease.

By “an amount sufficient” is meant the amount of a compound of the invention required to reduce PI3K activity in a cell, treat or prevent a C. albicans infection in a clinically relevant manner, treat or prevent an inflammatory condition in a clinically relevant manner, or treat or prevent a proliferative disorder in a clinically relevant manner. A sufficient amount of an active compound used to practice the present invention for therapeutic treatment of conditions caused by an inflammatory disease or proliferative disorder varies depending upon the manner of administration, the age, body weight, and general health of the patient. Ultimately, the prescribers will decide the appropriate amount and dosage regimen. The appropriate amounts for any therapy described herein can be determined from animal models, in vitro assays, and/or clinical studies. A sufficient amount of an active compound used to practice the present invention for inhibiting PI3K activity in a cell can be determined by using known in vitro assays, such as the assay of Example 36.

By “anticancer agent” is meant a compound that, individually, inhibits the growth of a neoplasm in a mammal. Anticancer agents that can be used in the conjugates of the invention include, without limitation, microtubule inhibitors, topoisomerase inhibitors, platins, alkylating agents, and anti-metabolites. Particular anticancer agents that are useful in the methods and compositions of the invention include, without limitation, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine. The ability of a compound to inhibit the growth of a neoplasm can be assessed using known animal models.

By “Candida albicans infection” is meant the invasion of a host animal by Candida albicans cells. For example, the infection may include the excessive growth of fungi that are normally present in or on the animal, or growth of fungi that are not normally present in or on the animal.

The term “inflammatory disorder” encompasses a variety of conditions, including autoimmune diseases, proliferative skin diseases, and inflammatory dermatoses. Inflammatory disorders can result in the destruction of healthy tissue by an inflammatory process, dysregulation of the immune system, and/or unwanted proliferation of cells. Examples of inflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Bebcet's disease; Bell's palsy; bullous pemphigoid; cerebral ischaemia; chronic obstructive pulmonary disease; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderna; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis.

The term “mammal” includes, without limitation, humans, cattle, pigs, sheep, horses, dogs, and cats.

As used herein, “non-naturally occurring” refers to a targeting group which is chemically synthesized (i.e., man made).

By “neoplasm” is meant a collection of cells multiplying in an abnormal manner. The term encompasses neoplastic cells located at the original site of proliferation (“primary tumor or cancer”) and their invasion of other tissues, or organs beyond the primary site (“metastisis”).

By “pharmaceutical composition” is meant a composition containing a compound of the invention, formulated with a pharmaceutically acceptable excipient, and manufactured in compliance with the rules of a governmental regulatory agency as part of a therapeutic regimen for the treatment or prevention of disease in a mammal. Pharmaceutical compositions can be formulated, for example, for intravenous administration (e.g., as a sterile solution free of particulate emboli and in a solvent system suitable for intravenous use), or any other formulation described herein.

By “proliferative disorder” is meant a disease characterized by the presence of a neoplasm or a proliferative skin disorder.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis. As will be appreciated by one skilled in the art, a particular disease, disorder, or condition may be characterized as being both a proliferative skin disease and an inflammatory dermatosis. An example of such a disease is psoriasis.

As used herein, “substantially pure” refers to a wortmannin conjugate composition that is less than 10%, 5%, 1%, or even 0.1% by mass unconjugated wortiannin-like compound or wortmannin conjugated to a different targeting group (i.e., a targeting group other than that of the wortmnannin conjugate). For example, the targeting group can be a material of biological origin which is isolated and purified prior to conjugation to a wortmannin-like compound to form a WmC20 conjugate of the invention.

By “targeting group” is meant a group of more than 300, 400, 500, 600, 800, or even 1,000 daltons or, if less than 300 daltons, a low molecular weight ligand having greater than 1×10⁴ M⁻¹, 1×10⁵ M⁻¹, 1×10⁶ M⁻¹, or even 1×10⁷ M⁻¹, affinity for an in vivo site within a mammal other than a site for which wortmannin has greater than 1×10⁴ M⁻¹ affinity, an anticancer agent, or an anti-inflammatory agent. Exemplary low molecular weight ligands include, without limitation, folate, methotrexate, and RGD peptides, among others.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

As used herein, “treating a proliferative disorder” refers to measurably slowing, stopping, or reversing the growth rate of the neoplasm or neoplastic cells in vitro or in vivo. Desirably, the slowing of the growth rate is by at least 20%, 30%, 50%, or even 70% in the presence of a conjugate of the invention in comparison to the growth rate in the absence of a conjugate of the invention, as determined using a suitable assay for detennination of cell growth rates.

As used herein, the terms “wortmannin C20 derivative” and “WmC20 derivative” refer to a wortmannin-like compound which is derivatized at the C20 position. For example, a wortmannin-like compound can be reacted with a linker bearing a primary amine, secondary amine, or thiol in a ring-opening reaction which conjugates the linker to position C20 of the wortmannin-like compound to form a wortmannin C20 derivative. The linker can be covalently attached to a targeting group. A general molecular scaffold of the wortmannin C20 derivative covalently tethered to a targeting group via a linker, referred to herein as a wortmannin conjugate or WmC20 conjugate, is shown below.

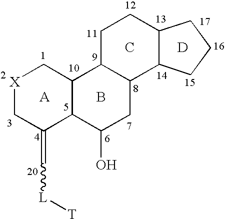

By “wortmannin-like compound” is meant a compound which inhibits PI3K by covalently binding to Lys-802. Wortmannin-like compounds include, without limitation, viridin, viridiol, demethoxyviridin, demethoxyviridiol, wortmannin, wortmannolone, 17-hydroxywortmannin, 11-desacetoxywortmannin, Δ9,11 dehydrodesacetoxywortmannin, and analogues and derivatives described herein.

### BRIEF DESCRIPTION OF THE DRAWINGS

FIG. 1 is a schematic drawing showing the mechanism of action of wortmannin and wortmannin-like compounds with the presumed molecular target PI3K. In the generally accepted mechanism of wortmannin action (bottom, dotted arrow), wortmannin binds to PI3K, with attack by a lysine in the ATP binding site of PI3K (see Wymann et al., Mol. Cell Biol. 16:1722 (1996); and Wipf et al., Org. Biomol. Chem. 2:1911(2004)). In contrast, without wishing to be bound by any theory, the WmC20 conjugates of the invention generate wortmannin through an intra-molecular attack (top) and the resulting wortmannin then binds the PI3K. As result WmC20 conjugates need not fit into the active site of PI3K. “S, N” indicates a sulfur or nitrogen can be at C20.

FIGS. 2A and 2B are graphs showing the effect of incubating selected compounds of the invention in PBS. FIG. 2A is a graph depicting the decay of wortmannin with and without biological amines. Instability is due to presence of biological amines. FIG. 2B is a graph depicting the stability of WmC20 derivatives 2a and 2b in the presence or absence of proline. Reaction of wortmannin at the C20 position yields derivatives (2a, 2b, examples 1, 2) that are resistant attack by proline, which accelerates wortmannin decay (2A). Data were fit to a single first order decay model with constants given in Table 1 and methods in example 34.

FIG. 3 depicts schemes for the synthesis of WmC20 derivatives and wortmannin conjugates. Synthesis through WmC20-linker intermediates (top)-Step 1: Synthesis of Wortmannin C20 Derivative W−L. Step 2: Conjugation of WmC20 derivative to T to yield W−L−T. Several component reactions can be used to complete Step 1 or Step 2. Synthesis through linker-targeting group intermediates (bottom)—Step 1: Synthesis of a linker-targeting group (L−T). Step 2: Synthesis of the WmC20 conjugate W−L−T.

FIG. 4 depicts a scheme for the synthesis of WmC20 conjugates through the W−L intermediates 2a and 2b. Syntheses of 2a, 2b, 3a and 3b are given in examples 1-4. Some examples of WmC20 conjugates synthesized with this chemistry are examples 12-18.

FIG. 5 is a schematic drawing showing the synthesis of WmC20 conjugates through linker-targeting agent (L−T) intermediates. The targeting group is within the dotted enclosure. The structure between the targeting group and wortmannin is the linker. Structures are from examples 19 and 22.

FIGS. 6A-6D depict HPLC curves demonstrating the variable generation of wortmannin from WmC20 derivatives modified at C20 with tertiary amines. (6A) Formation of the wortmannin from 2a (example 1) with tertiary amine at C20. The internal standard is MHB (methyl-4-hydroxybenzoate). (6B) Undetectable formation of wortmannin from 2b. (6C) Formation of wortmannin from WmC20 conjugate of example 12, which features a tertiary amine at C20. (6D) Slow or undetectable formation of wortmannin from example 13. Compounds with a tertiary amine at C20 produce wortmannin faster than those with a secondary amine at C20. HPLC system 3 was used for (A) and (C) while HPLC system 4 was used for (B) and (D) (see example 38).

FIG. 7A is a series of graphs showing the time course of wortmannin 1 generation from WmC20 conjugates. The time course of wortmannin generation and degradation was monitored by HPLC.

FIG. 7B depicts the model used to analyze the data of FIG. 7A. The model yields equation 2 (see Example 35). Values of k1 and k2 are given in Table 2.

### DETAILED DESCRIPTION

The invention features wortmannin conjugates and their use as inhibitors of PI3K activity, anti-proliferative agents, anti-inflammatory agents, and anti-Candida albicans agents. The wortmannin conjugates described herein have three characteristic components: a WmC20 derivative (W) covalently tethered, via a linker (L), to a targeting group (T).

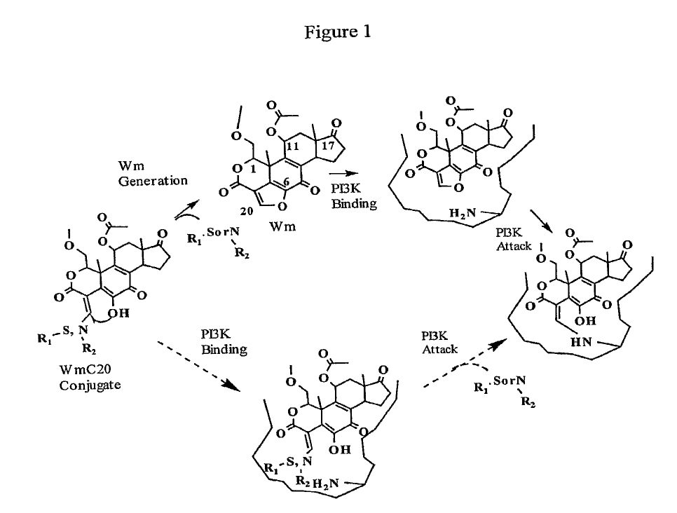

The wortmannin conjugates of the invention can exhibit three key features: (i) they are stabilized against attack at C20 by common amines present in biological samples and, therefore, overcome tile instability problems associated with wortmannin-like compounds; (ii) they spontaneously release the parent wortmannin-like compound in vivo through an intra-molecular attack at C20 by hydroxyl group at C6 to reform the furan ring, see FIG. 1; and (iii) through the conjugation of targeting groups to WmC20 derivatives, the wortmannin conjugates of the invention achieve a biodistribution, pharmacokinetic profile, and/or localized concentration that cannot be achieved with unconjugated wortmannin-like compounds. Each of these features is described in more detail below.

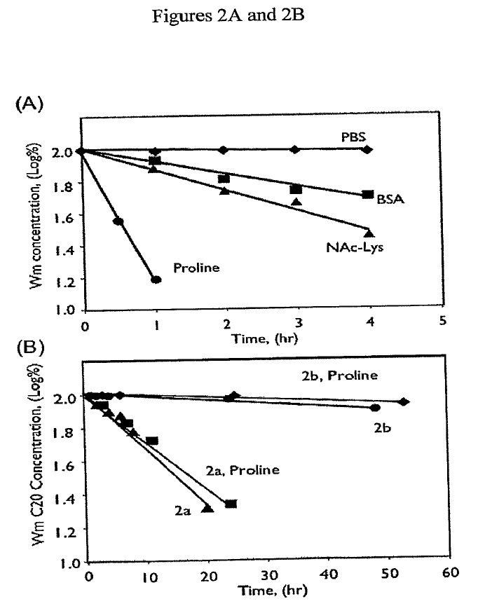

1. Stability in biological media: The wortmannin conjugates of the invention are stabilized because they are protected against attack at the C20 position by nucleophiles, e.g. amines, thiols, amino acid side chains of proteins, present in biological samples. For example, the half-lives for Wortmannin in RPM and MEM tissue culture media, are about 10 minutes (see Holleran et al., Anal. Biochem. 323:19 (2003)). In contrast wortmannin compounds with secondary amine linkers at C20 have half-lives longer than 100 hours and this value is independent of the presence of nucleophiles, see FIG. 2B and Table 1.

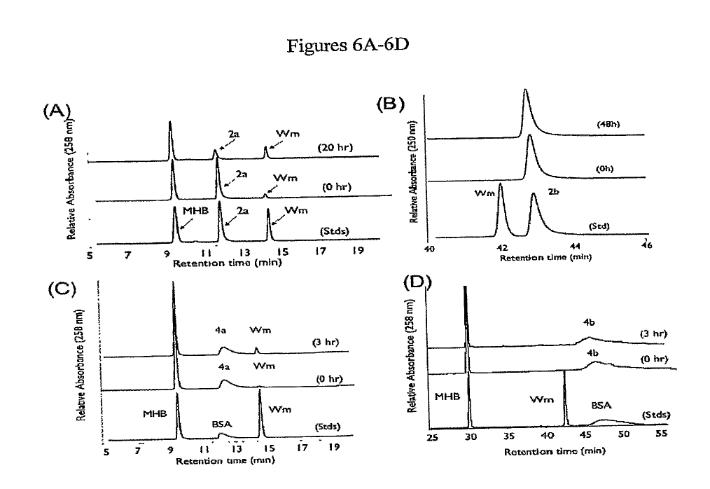

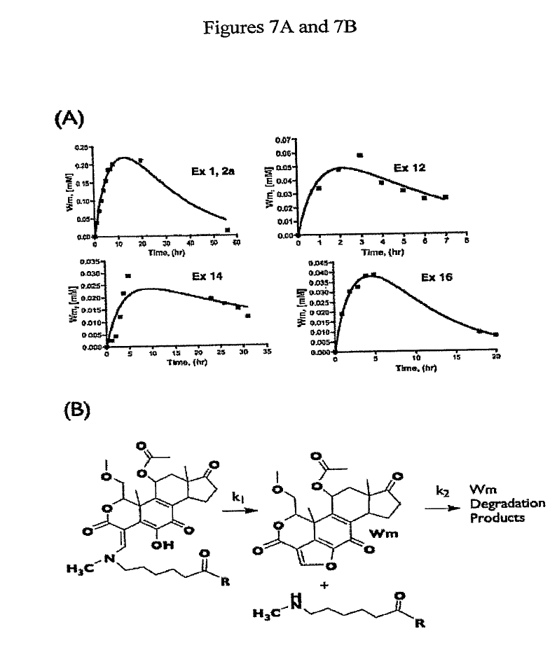

2. Activity through wortmannin release: The wortmannin conjugates of the invention release or generate wortmannin or the parent wortmannin-like compound through an intra-molecular attack of the hydroxyl group at C6 to C20 to reform the furan ring, see FIG. 1. This intra-molecular attack releases wortmannin-like compound even in organic solvents, indicating that in aqueous media the water does not have to participate in this transformation (see Yuan et al., J. Med. Chem. 49:740 (2006)). One result of the intramolecular nature of this transformation is that it is the nucleophile at C20 which controls the release rate of wortmannin-like compound from the WmC20 conjugates of the invention. In biological systems, enzymes or cofactors maybe present and produce more rapid rates of release than occur in controlled situations in vitro. Evidence for the generation of wortmannin by WmC20 conjugates is provided in FIGS. 6 and 7 and Tables 2-4.

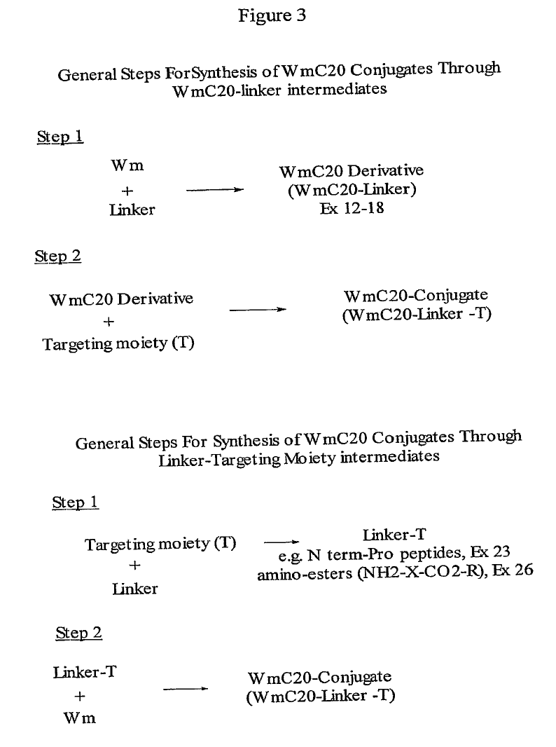

3. Diversity in physical and biological properties and targeting principles. The release mechanism shown in FIG. 1 permits pharmaceutical design, (the design of compounds exhibiting the general W−L−T structure of FIG. 3) based on a compound's fate (adsorption, tissue distribution), and without regard to the compound's ability to conform to structure/activity relationships that are crucial to pharmacological activity with conventional medicinal chemistry. Thus, the WmC20 conjugates can achieve a diversity of physical properties and pharmacological properties, including different biodistributions, pharmacokinetic patterns, or local concentrations. Based on appropriate selection of targeting moieties (and to a lesser extent linkers), the compounds of the invention can be designed to achieve (i) a selective biodistribution based on molecular targeting, (ii) blood half-life control (due to varied size or albumin binding), (iii) sustained and local release from implants, (iv) enhanced potency of pharmacological agents used as targeting moieties, (v) a wide variety of physical properties. The diversity of physical properties obtained with WmC20 conjugates is summarized in Table 5b.

### Wortmannin-like Compounds

Wortmannin-like compounds feature a reactive center at C20 which chemically modifies a lysine residue in the active site of PI3K. Wortmannin-like compounds include, without limitation, viridin, viridiol, demethoxyviridin, demethoxyviridiol, wortmannin, wortmannolone, 17-hydroxywortmannin, 11-desacetoxywortmannin, and Δ9,11 dehydrodesacetoxywortmannin.

Wortmannin is a commercially available natural product whose production is described in U.S. Pat. No. 6,703,414, incorporated herein by reference.

A number of modified wortmannins and wortmannin-like compounds can be employed in the compositions and methods of the invention provided that they have an intact furan ring so that C20 can be reacted with the linkers. The reaction of the furan ring C20 is essentially independent of modification at many other positions on the wortmannin molecule.

Once wortmannin, viridin, viridiol, demethoxyviridin, and demethoxyviridiol, or other PI3K inhibitors are isolated and purified, analogs of each may be prepared via well known methods as described, for example, in U.S. Pat. No. 5,726,167 (see, also, Grove et al., J. Chem. Soc. 3803 (1965); Hanson et al., J. Chem. Soc. Perkin Trans. 11311 (1985); and Aldridge et al., J. Chem. Soc. Perkin Trans. 1943 (1975).

A variety of wortmannin-like compounds are obtained by chemically modifying the starting material. Generally, the Cl hydroxy functionality may be acetylated, alkylated, oxidized, or dehydrated, acylated, and alkylated. Similarly, the C17 ketone functionality may be alkylated, or reduced to form an alcohol. The C3 hydroxy functionality may also be alkylated or acylated (see U.S. Pat. No. 5,726,167). 11-substituted, 17-substituted and 11, 17 disubstituted derivatives of wortmannin can be prepared as described in U.S. Pat. No. 5,480,906 and Creemer et al., J. Med. Chem. 39:5021 (1996). The conversion of the carbonyl at C17 of wortmannin to a hydroxyl group leads to 17-hydroxy wortmannin, which has enhanced affinity for PI3K than native wortmannin (see Norman et al., J. Med. Chem. 39:1106 (1996)).

Other wortmannin-like compounds that can be used in the methods and compositions of the invention include alpha/beta-viridin, 1-acetylviridin, 1-methylether of viridin, demethoxyviridin, demethoxyviridin mono-acetate, dehydroxyviridin, demethoxyviridin mono-methanesulfonate, diacetyldemethoxyviridol, viridiol, 1-O-acetylviridiol, 1-O-methyl-methylether of viridiol, demethoxyviridiol, 1-acetyldemethoxyviridiol, and 1-O-methylether dimethoxyviridiol (see, U.S. Pat. No. 5,726,167).

### Linkers

The linkers (L) have the general structure of: (i) a primary amine, secondary amine, or thiol for reaction at C20 of a wortmannin-like compound, (ii) a bridging structure, and (iii) a reactive group distal to the primary amine, secondary amine, or thiol for attachment to the targeting group (T). The bridging structure can be any of a variety of chemical structures (e.g., straight chain, branched alkyl, or aryl, and each substituted with other atoms), but in all instances contains the required amine or thiol group.

The major functions of the linking group are (i) to provide primary or secondary amine or thiol at C20 which controls the rate of wortmannin regeneration (thiol faster than secondary amine faster than primary amine), (ii) to provide a functional group for reaction with T, (iii) to confer physical properties on W—L (or L—T) facilitating the synthesis of the desired W—L—T. A key physical property required for synthetic reactions is the solubility of all reactants in a single solvent.

For example, the linker can include a terminal carboxyl group that can be attached to a primary amine of a targeting group (T). Attachment chemistries that can be used to achieve covalent attachment include activation of the carboxyl group with a N-hydroxysuccinimide ester (synthesized with carbodiimide and N-hydroxysuccinimide), followed by reaction with, for example, amine groups contained on T. A wide variety of conjugation reactions can be used to attach WmC20 derivatives to targeting groups. Furthermore, the linker can have multiple reactive sites for the attachment of multiple targeting groups to produce multivalent wortmannin conjugates. Desirably, the linker is the side chain of an amino acid that is part of a protein or peptide. Examples of linkers are given in Tables 6a and 6b.

The linker component of the invention is, at its simplest, a bond between a WmC20 derivative and a targeting group. Typically, the linker provides a linear, cyclic, or branched molecular skeleton having pendant groups covalently linking a wortmannin derivative to a targeting group.

So-called zero-length linkers, involving direct covalent joining of the C20 position of a wortmannin-like compound with a reactive moiety of the targeting group without introducing additional linking material may, if desired, be used in accordance with the invention. For example, a wortmannin-like compound can be reacted directly with a primary amine (e.g., a lysine side chain), secondary amine (e.g., a proline, or histidine side chain), or thiol (e.g., a cysteine side chain) of a protein or peptide to form a wortmannin conjugate of the invention in which the linker is a single bond.

Most commonly, however, the linker will include two or more reactive moieties, as described above, connected by a spacer element. One of the reactive moieties will be derived from a primary amine, secondary amine, or thiol for covalent attachment to the C20 position of the wort in-like compound. The presence of such a spacer permits bifunctional linkers to react with specific functional groups within the wortmannin derivative and the targeting group, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the wortmannin derivative and the targeting group.

Spacer elements in the linker typically consist of linear or branched chains and may include a C₁₋₁₀ alkyl, a C₁₋₁₀ heteroalkyl, a C₂₋₁₀ alkene, a C₂₋₁₀ alkyne, C₅₋₁₀ aryl, a cyclic system of 3 to 10 atoms, or —(CH₂CH₂O)ₙCH₂CH₂—, in which n is 1 to 8.

In some instances, the linker is described by formula IIa:

G²-(X¹)—(R₁₀)-(Z²)ₛ-(Y¹)ᵤ-(Z¹)ₒ-G¹ (IIa)

In formula IIa G¹ is a bond between the linker and the targeting group; G² is a bond between the linker and the WmC20 derivative; X¹ is S or NR₁₁; each of Z¹ and Z² is, independently, selected from O, S, and NR₁₂; each of o, s, and u is, independently, 0 or 1; Y¹ is selected from carbonyl, thiocarbonyl, sulphonyl, and phosphoryl; R₁₂ is selected from hydrogen, C₁₋₁₀ alkyl, C₂₋₁₀ alkene, C₂₋₁₀ alkyne, and C₅₋₁₀ arylgroup; R₁₁ is selected from hydrogen, C₁₋₁₀ alkyl, C₂₋₁₀ alkene, C₂₋₁₀ alkyne, and C₅₋₁₀ arylgroup; and R₁₀ is a C₁₋₁₀ alkyl, C₁₋₁₀ heteroalkyl, C₂₋₁₀ alkene, a C₂₋₁₀ alkyne, a C₅₀₁₀ aryl, a cyclic system of 3 to 10 atoms, or —(CH₂CH₂O)_{q}CH₂CH₂—in which q is an integer of 1 to 8; or R₁₀ and R₁₁ combine to form a cyclic system of 3 to 10 atoms.

For example, linkers of the present invention can be formed by reaction of a primary amine, secondary amine, or thiol with the C20 position of a wortmannin-like compound. The amine or thiol contains a bridging structure and a second functional group for attachment to the targeting group. Useful linkers can be made from amino acids using the methods described in the Examples.

Linkers can also have multiple reactive sites for the attachment of multiple targeting groups, such that the wortmannin conjugate is multivalent.

The rate of the intra-molecular attack, and wortmannin-like compound generation, varies greatly with the nature of the linker and the chemistry of the targeting moiety. In the case where a thiol of L is reacted with C20, the liberation of wortmannin is faster than the release shown for any of the compounds in FIGS. 6 and 7 and in Table 2. When the secondary amine of L is used to modify C20, the rate of release is moderate and varies with targeting group as well as the linker (see Table 2). These rates are the apparent rates measured under the specified conditions. When a primary amine of L is used, the release of wortmannin is extremely slow, so that no release was detectable under the conditions employed as shown in FIGS. 6B and 6D.

The variable release rates for wortmannin-like compounds that can be achieved with the wortmannin conjugates pen-nit their use in diverse pharmaceutical applications. For example, with implantable devices a slow release formulations may be preferred and here a secondary amine linkage at C20 employed. Alternatively, in acute care applications, a faster release of wortmannin is needed (e.g., the intranasal administration of wortmannin conjugate for an asthma attack might employ a wortmannin conjugates made by reaction of a thiol at the C20 of the wortmannin-like compound).

### Targeting Groups

For use in the wortmannin conjugates of the invention, the targeting group must contain a reactive group for reaction with the linking group. Suitable reactive groups include, without limitation, carboxylic acid, hydroxyl, sulfhydryl, and amino groups. Where desired, the functional groups in the targeting group may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; conversion of alcohols to thiols using reagents such as tosyl chloride followed by transesterification with thioacetate; and hydrolysis to the thiol with sodium acetate.

The targeting group can be used to adjust the receptor binding, biodistribution, pharmacokinetics, oral bioavailability, protein binding, solubility, stability or general physical properties of the WmC20 conjugate as desired. Five non-limiting and possible functions of targeting groups are given below.

1. Local Release: The wortmannin conjugates of the invention can be used as implants. An implant is a therapeutic agent which is injected into a specific anatomical location and which produces a locally high concentration of a therapeutic agent at the injection site. Using this approach the targeting group is an implant, such as a solid material, slow release solid phase, wafer, bead (see example 16), stent, or microsphere, covalently tethered to a WmC20 derivative via a linker. Following implantation, the release of wortmannin-like compound is controlled by chemistry selected (e.g., primary amine, secondary amine, or thiol) for the synthesis of the WmC20 conjugate. The targeting group can include a radioactive isotope to exploit the synergism between radiotherapy and wortmannin. Implantable radioactive beads are employed in the treatment of prostate cancer and a synergism between radiation therapy and wortmannin has been noted (see Price et al., Cancer Res. 56:246 (1996)). Alternatively, the wortmannin conjugate of the invention is not covalently attached to the implant, but releases over some period of time wortmannin-like compound and/or WmC20 conjugate.

2. Molecular Targeting: The targeting groups can be selected to permit the wortmannin conjugates to be transported through the vascular system and bind to a specific molecular target, resulting in the selective accumulation of wortmannin at the target site of interest. Examples of this type of targeting group include, without limitation, antibodies (examples 14, 15, 25, 26, and 27), antibody fragments, proteins (examples 12, 13, 19, 20, and 21), peptides (examples 22, and 23), peptidomimetics, and low molecular weight ligands for receptors (example 24). Examples of molecular targets include differentiation antigens, growth factor receptors, integrins, cell matrix components, G-protein coupled (7TM) receptors, and lipid head groups, and mucopolysaccharides (see, for example, Garnett et al., Adv. Drug Deliv. Rev. 53:171 (2001); and Sezaki and Hashide “Macromolecule-drug conjugates in targeted cancer chemotherapy” in CRC Critical Reviews in Therapeutic Drug Carrier Systems (1984) p. 1).

3. Blood half-life control. The targeting group can also be a synthetic or man-made polymer, such as polyethylene glycol, or a natural product derived polymer, such as albumin, dextran, and hydroxyethyl starch. Such polymers do not bind to a known molecular target but alter the biodistribution and/or pharmacokinetic profile of the WmC20 conjugate (examples 12, 13, 17, and 18) in comparison to the unconjugated wortmannin-like compound. Targeting can be achieved through the enhanced permeability and retention effect (EPR effect) due to a lack of a tumor lymphatic system (see Duncan et al., Nat. Rev. Drug Discov. 2:347 (2003); and Seymour et al., Crit. Rev. Ther. Drug Carrier Syst. 9:135 (1992)). Polymeric targeting groups can be used to attain a desired blood half-live, such by selecting a dextran of an appropriate size Kaneo et al., Biol. Pharm. Bulletin 20:181 (1997)). Blood half control can also be achieved by using hydrophobic targeting groups, which maximize protein binding.

4. Potency enhancement of WmC20-conjugates: The targeting group can function to enhance the pharmacological activity of the conjugates of the invention. Conversely, the attachment of the wortmannin-like compound to an existing pharmacological agent when used as a targeting group can enhance the potency of the pharmacological agent In this regard the targeting group can be anticancer agent (e.g. methotrexate, paclitaxel), an anti-inflammatory agent (e.g., a corticosteroid), or an anti-fungal agent. In the case of anti-cancer agent, the conjugate can be used for the treatment of cancer and/or inhibiting the formation of metastases. Desirably, the conjugates reduce the minimum efficacious dose of the existing clinical regimen for the anticancer agent administered as a monotherapy (e.g., paclitaxel for the treatment of breast cancer). The benefit to the patient is an increase in the therapeutic index of the anticancer agent administered as a wortmannin conjugate in comparison to administration as a monotherapy (see examples 30-33).

5. Alter physical properties: The targeting can make the WmC20 conjugate more hydrophilic, hydrophobic, positively charged, negatively charged, larger, water soluble, or stable (examples 25-29). The targeting group can be chosen to increase or regulate the speed of oral bioavailability or binding to plasma proteins.

Accordingly, the wortmannin conjugates of the invention are, by virtue of the targeting group selected, an improvement over existing therapeutic regimens and can be used to reduce adverse drug reactions, extend the life of a patient, and/or improve the cure rate.

### Wortmannin Conjugates

As shown in the scheme of FIG. 1, the conjugates release wortmannin first through an intra-molecular attack at the C20 position, thereby releasing the linker and targeting molecule, before reaction in the PI3K binding pocket. Because the intra-molecular attack model does not require the wortmannin conjugate to bind to the ATP site of PI3K, the linker and targeting group can have any size or physical character. This permits the attachment of a wide variety of targeting groups to wortmannin that can afford a number of improved properties compared with earlier wortmannin based compounds. These include (i) improved the stability (relative to wortmannin) in various media, (ii) reduced toxicity to cells other than those where a high wortmannin concentration is desired (non-target cells), (iii) targeting of wortmannin to cells of interest through the binding activity of a targeting group, and (iv) providing a means of attachment to solid supports, to permit slow release of wortmannin in a particular region of interest (e.g., a support on an implantable medical device). In each case, the generation of wortmannin by the intra-molecular attack yields the wortmannin, the active drug.

Wortmannin conjugates of the invention can be described, for example, by any of formulas IIIa-IIIi, which vary in the identity of the wortmannin-like compound employed: Viridin (IIIa), viridiol (IIIb), demethoxyviridin (IIIc), demethoxyviridiol (IIId), wortmannin (IIIe), wortmannolone (IIIf), 17-hydroxywortmannin (IIIg), 11-desacetoxywortmannin (IIIh), and Δ9,11 dehydrodesacetoxywortmannin (IIIi).

There are several general considerations in the selection of the linkers, targeting groups, and reactions that can be used in the synthesis of the wortmannin conjugates of the invention. For all reactions, conditions must be used which preserve W, that is, which preserve the key structural features of the wortmannin-like molecule that are essential for biological or pharmacological activity. Essential and somewhat labile features of these molecules include the carbonyl atoms at the C3 and C7 carbons, and the unstaturated bonds at various positions for the various wortmannin-like structures. The lactone ring of wortmannin is particularly sensitive to strong base and when broken yields a pharmacologically inactive material. The unstaturated rings and carbonyl groups can fail to survive strong reducing agents. Second, since the linker L must have two reactive groups, one for reaction with the C20 of Wm and for reaction with T, the formation of unwanted dimers can occur. Thus, when reacting W with L, the formation of the dimer W—L−W must be avoided. Similarly, if L and T are reacted to obtain L−T, followed by reaction with W to obtain W—L−T, formation of the dimer T—L—T must be avoided. In light of these considerations there are three general and preferred methods of avoiding unwanted dimers illustrated with the formation of W−L.

1. Differential reactivity: Using linkers with two reactive groups, but with different activity can be done, as is the case for amine and a carboxyl group L's which allow the amines to react with the C20 of the wortmannin-like compound, followed by reaction of the carboxyl group with T.

2. Use of a large excess of L: A large excess of L over W can be used, followed by removal of unreacted L. This strategy can be used to advantage with the linker diamines like hexane diamine which have good solubility and are cheap.

3. Use of protecting groups: A linker with one reactive group protected, and which can be deprotected in a manner consistent with the needs above can be used (see examples 8, 9, 28, and 32).

As shown in FIG. 3, WmC20 conjugates can be synthesized through W−L: (W+L=W−L, then W−L+T=W−L—T). The synthesis of WmC20 conjugates can also be synthesized through L—T: (L+T=L−T, then W+L−T=W−L−T).

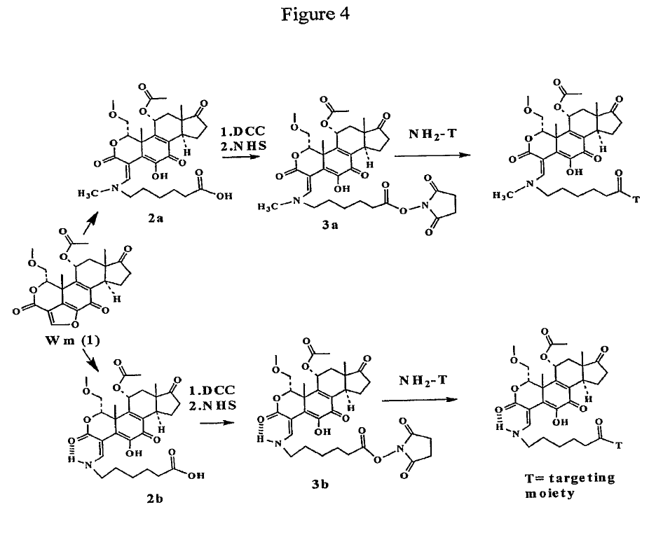

One method for the synthesis of WmC20 conjugates using W−L is the reaction of wortmannin-like compound in organic media with 1-10 moles of the amine or thiol linker which further includes a carboxylic acid for reaction with a targeting group. The resulting product is a WmC20 derivative having an amino or thioether group at C20. Natural or unnatural amino acids, diamines, amino-thiols can be used as linkers. The WmC20 derivative is then purified to remove unreacted linker. The carboxyl group of the WmC20 derivative can then be reacted with activating agent(s), such as carbodiimide and N-hydrosuccinimide, to yield an N-hydroxysuccinimide ester, that reacts with the amino groups of a targeting molecule, T (see FIG. 4). This general strategy is used in examples 12-18. However, a wide variety of conjugation reactions can be used to attach WmC20 derivatives to targeting moieties.

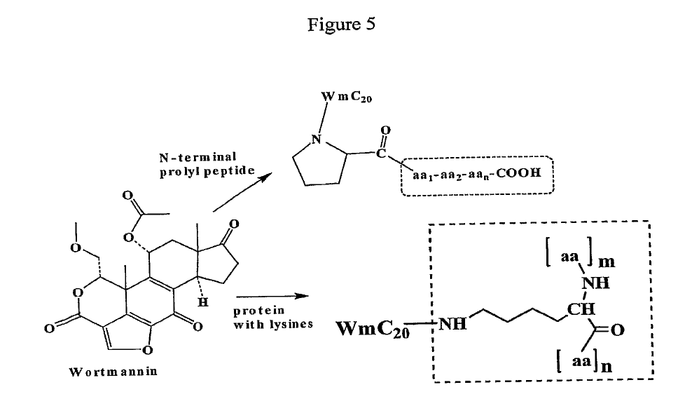

Another method for the synthesis of WmC20 conjugates utilizes L−T wherein T is a linear peptide with an N-terminal proline, as shown in FIG. 5 and described in example 22. Here the proline linker is attached to the amino acid residues of the linear peptide (T) during solid phase peptide synthesis. Alternatively, synthesis via L−T includes the reaction of a wortmannin-like compound with the episilon amino group of a protein or peptide, as described in example 19. Here the linker is a bond with the nitrogen atom of a lysine residue within the protein. Multiple lysine residues in a protein may serve as multiple sites for conjugates containing multiple WmC20 conjugates in a single protein.

A feature of the invention is the ability to achieve highly variable rates of intra-molecular attack and wortmannin generation. The rate of the intra-molecular attack is principally controlled by the nature of the groups reacted with C20 and is thiol (fastest), tertiary amine (moderate), and primary amine (slow). Various rates of release of wortmannin are useful in different applications. When WmC20 derivatives are covalently coupled to a solid phase implant (T), a slow release of wortmannin-like compound (weeks to months) can be useful. This rate of release can be slower than measured by HPLC based analytical methods used to monitor wortmannin release (FIG. 6). Alternatively, in acute care applications, a faster release of Wortmannin is needed. For example, the intranasal administration of WmC20 conjugate for an asthma attack might employ a WmC20 conjugates having a thioether at position C20.

Wortmannin Release by the WmC20 Conjugates of the Invention

A variety of lines of evidence support the release of wortmannin by WmC20 derivatives and WmC20 conjugates. Our data supports the conclusion that wortmannin release is principally governed by the nature of the group at C20 and is independent of the targeting group.

HPLC data: The release of wortmannin from the conjugates given in examples 12, 14 and 16 was evident on HPLC chromatograms, (FIG. 6C) and is summarized in FIG. 7A. HPLC were fit to a kinetic model of wortmannin release shown in FIG. 7B with constants provided in Table 2. Data indicate that WmC20 conjugates with a tertiary amine reacted at C20 release wortmannin faster than those with a secondary at group at C20. Wortmannin release for the WmC20 derivative example 2 and the WmC20 conjugates made according to examples 13, 15, and 18 could not be detected by the HPLC method.

Inhibition of PI3K enzyme activity: According to the wortmannin release model shown in FIG. 1 (top), the abilityWortmanninC20 conjugates to release wortmannin, that is the rate of release, should parallel their ability to inhibit PI3K activity in assay protocols which use relatively short incubation times. Wortmannin C20 conjugates that release wortmannin rapidly, like those with a tertiary amine at C20, should inhibit PI3K, while those with a secondary amine at C20 should release far less wortmannin (more slowly). Table 4 indicates the 1C50's (concentrations necessary for inhibition of 50% enzyme activity) of wortmanninC20 tertiary amines (examples 1, 12, and 14) was far lower than the corresponding secondary amines (examples 2, 13, and 15), indicating this prediction of the wortmannin release model is correct.

Inhibition of cell proliferation: According to the wortmannin release model, the ability of WmC20 conjugates to release wortmannin should parallel their ability to inhibit cell proliferation. WmC20 conjugates were therefore assayed for their anti-proliferative activity against A549 cells and HeLa cells as shown in Table 4. Table 4 indicates that the IC50's (concentrations required to give a 50% inhibition of cell proliferation) were far lower with wortinannin's modified at C20 by attachment of a tertiary amine (examples 1 and 17) than those modified with a secondary amine (examples 2, 18). In addition, attachment of 2a or 2b to a 40 kDa dextran carrier had little effect on the IC50. This indicates that the nature of the amine reacted at C20 determines the activity of the conjugate, consistent with the greater release of wortmannin with tertiary amines at C20 evident by HPLC. However, Table 4 also indicates that WmC20 conjugates featuring a secondary amine at C20 (examples 2 and 18) have anti-proliferative activity. This implies the release of wortmannin from wortmannin secondary amine conjugates occurs at a slow rate in a cell based system, and/or in manner that was not duplicated by in vitro experiments. In vitro experiments are given in Table 2 (release assessed by HPLC) and Table 3, (release assessed by PI3K inhibition).

Further details of the synthesis of wortmannin conjugates can be found in the Examples.

### Therapy

The wortmannin conjugates of the invention are preferably administered by a subcutaneous, intravenous or intraperitoneal injection. The conjugates can be formulated as known in the art using physiologically acceptable buffers, preferably phosphate or citrate, with or without a tonicity-adjusting agent, such as NaCl, or mannitol. Preservatives suitable for use with parenteral products such as thimersol can be added. For parenteral administration into humans, conjugates must be sterile, with preferred methods of sterilization being terminal sterilization (heat) or passage through 220 nm filters. In a preferred method of formulation for parenteral products, WmC20 conjugates are filter sterilized and lyophilized for enhanced stability. With lyophilization the addition of excipients maybe necessary for stability in the lyophilized state, excipients including polymers like dextrans or sugars. Reconstitution can be with a buffer or an isotonicity conferring, steril, aqueous solution. Preferred examples include 0.1M saline, phosphate buffered saline or mannitol (0.3M).

The wortmannin conjugates of the invention can also be administered in conjunction with a wide variety of implantable devices (i.e., such as those described by Langer et al., Pharmacol. Ther. 21:35 (1983); Mainardes et al., Curr. Drug Targets 5:449 (2004); Hatefi et al., J. Control Release 80:9 (2002); Westphal et al., Neuro-oncol 5:79 (2003); and Moul et al., Urol. Nurs. 21:385 (2001)). In some cases the WmC20 conjugate will be released by the implant, diffuse from the implant and then generate wortmannin-like compound. In some cases WmC20 derivatives will be covalently attached to the implant and will release wortmannin-like compound which will diff-use from the implant. It is desirable that implantable drug release devices provide locally high concentrations of WmC20 conjugates or wortmannin-like compound over a prolonged period of time. Implants can be made of from, for example, biodegradable polymers that can be used to synthesize into microspheres or gels. The conjugates can also be used to coat implants like stents or administered from osmotic pumps (see Halkin et al., J. Interv. Cardiol. 17:271 (2004)).

Wortmannin conjugates can be administered locally or systemically to decrease inflammatory, immune responses, reduce cell proliferation, or to treat C. albicans infections.

Therapeutic formulations may be in the form of liquid solutions or suspensions; for oral administration, formulations may be in the form of tablets or capsules; for ocular administration, formulations may be in the form of eye drops; for topical administration, formulations may be in the form of creams or lotions; and for intranasal formulations, in the form of powders, nasal drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

Wortmannin conjugates may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, calcium, sodium, potassium and the like.

Administration of wortmannin conjugates in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release of the wortmannin conjugate. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided in a single unit dosage form, such as chewable tablets, or as hard gelatin capsules wherein the active ingredient is mixed with an inert solid diluent, or as soft gelatin capsules wherein the active ingredient is mixed with water or an oil medium.

Pharmaceutical formulations of the wortmannin conjugates described herein include isomers such as diastereomers and enantiomers, mixtures of isomers, including racemic mixtures, salts, solvates, and polymorphs thereof.

The exemplary dosage of wortmannin conjugate to be administered will depend on such variables as the type and extent of the disorder, the overall health status of the patient, the therapeutic index of the selected targeting group and wortmannin-like compound, and their route of administration. Standard clinical trials may be used to optimize the dose and dosing frequency for any particular conjugate of the invention.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1

### Synthesis of wortmannin C20-N(Me)-hexanoic acid derivative

Wortmannin (42.8 mg, 0.1 mmol), 6-(N-methylamino)hexanoic acid hydrogen chloride, referred to as N(Me)-hexanoic acid.HCl, (90 mg, 0.5 mmol), and triethylamine (20 μl) were mixed in anhydrous DMSO (1 mL). The mixture was stirred at room temperature till complete as shown by TLC. The final solution was purified by reverse phase HPLC with acetonitrile and water as solvents. The injection mixture was made by mixing reaction mixture with acetonitrile (containing 50% water) by 1:1 ratio just before the injection. A yellow powder was obtained following lyophilization. 39.5 mg, 68.9%. MS: C₃₀H₃₉NO₁₀, cal. 574.2652 (M+H⁺), found 574.2648; 1H NMR, (CDCl₃): 0.851 (3H, s, C18-CH₃), 1.412-1.540 (2H, m, CH₂CH₂CH₂—), 1.577 (3H, s, C19-CH₃), 1.650-1.780 (4H, m, —CH₂CH₂CH₂—), 1.797-1.845 (1H, dd, J1=14.8 Hz, J2=4.63 Hz, H-12), 2.063 (3H, s, CH₃CO), 2.090-2.200 (1H, r, H-16), 2.220-2.327 (1H, m, H-15), 2.370-2.426 (3H, m, H-16, CH₂CH₂COOH), 2.539-2.610 (1H, m, H-12), 2.794 (3H, broad, NCH₃), 2.907-3.030 (2H, m, H-14; CH₂OCH₃), 3.116-3.243 (2H, m, H-15, CH₂OCH₃), 3.254 (3H, s, CH₃O), 3.387-3.560 (2H, m, broad, NHCH₂), 4.051 (2H, broad, OH), 4.485 (1H, dd J1=2 Hz, J2=7.6 Hz, H-1), 6.063 (1H, dd, J1=4.8 Hz, J2=7.2 Hz, H-11), 8.177 (1H, s, H-20). This compound is 2a, see FIG. 4.

### Example 2

### Synthesis of wortmannin C20-NH-hexanoic acid

Wortmannin (42.8 mg, 0.1 mmol), 6aminohexanonic acid, referred to as 6-NH-hexanoic acid (65.5 mg, 0.5 mmol) and triethylamine (20 μu) were mixed in anhydrous DMSO (1 mL). The mixture was stirred at room temperature till complete (TLC monitoring). The final solution was purified by reverse phase HPLC with acetonitrile and water as solvents. The injection mixture was made by mixing reaction mixture with acetonitrile (containing 50% water) by 1:1 ratio just before the injection. After lyophilization, a yellow powder is obtained. 42.5 mg, 76.0%. MS: C₂₉H₃₇NO₁₀, cal. 560.2495 (M+H⁺), found 560.2499; 1H NMR, (CDCl₃): 0.828 (3H, s, C18-CH₃),1.430-1.507 (2H, m, —CH₂CH₂CH₂—), 1.532 (3H, s, C19-CH₃), 1.677-1.751 (4H, m, —CH₂CH₂CH₂—), 1.859-1.904 (1H, dd, J1=14.8 Hz, J2=2.8 Hz, H-12), 2.050 (3H, s, CH₃CO), 2.238-2.373 (3H, m, H-15, H-16),2.416 (2H, t, J=7.4 Hz, CH₂CH₂COOH), 2.542-2.629 (1H, m, H-12), 2.835-2.880(1H, m, CH₃OCH₂), 2.944-2.989 (1H, m, H-14),3.137-3.259 (2H, m, H-15, CH₃OCH₂), 3.275 (3H, S, CH₃OCH₂), 3.417 (2H, q, J1=13.0 Hz, J2=6.5 Hz), 4.328-4.345 (1H, m, H-1), 4.840 (2H, broad, OH), 5.982-6.009 (1H, m, H-11), 8.553 (1H, d, J=13.9 Hz, H-20), 9.810-9.920 (1H, m, NH). ¹³C NMR: 16.839, 21.143, 22.593, 24.287, 26.049, 26.431, 30.452, 33.619, 36.710, 38.594, 42.356, 42.410, 50.041, 59.419, 67.294, 73.330, 81.373, 88.431, 129.058, 137.093, 137.566, 151.134, 159.627, 166.403, 170.272, 178.010, 178.689, 218.285. This compound is 2b, see FIG. 4.

### Example 3

### Synthesis of the N-hydroxysuccinimide ester of wortmannin C20-N(Me)-hexanoic acid

Compound 2a (15.8 mg, 0.0276 mmol) was mixed together with N-hydroxysuccinimide (38 mg, 0.33 mmol) and DCC (68.2 mg, 0.33 mmol) in anhydrous acetonitrile (1 mL). The mixture was stirred at room temperature for 30 minutes. The solid was filtrated off and the residue was washed by anhydrous acetonitrile. The solvent was removed under reduced pressure. The residue was fractioned by silica gel chromatography with anhydrous ethyl acetate: hexane (5:1-10:1). A light yellow solid was obtained by precipitation with anhydrous hexane from trace amount of anhydrous ethyl acetate. 17 mg, 92%. C₃₄H₄₂N₂O₁₂, cal. 671.2816 (M+H⁺), found 671.2819. This compound is 3a, see FIG. 4.

### Example 4

### Synthesis of N-hydroxysuccinimide ester of wortmannin C20-NH-hexanoic acid

Compound 2b (11.2 mg, 0.02 mmol) was mixed together with N-hydroxysuccinimide (27.6 mg, 0.24 mmol) and dicyclohexylcarboimide (DCC) (49.5 mg, 0.24 mmol) in anhydrous acetonitrile (1 mL). The mixture was stirred at room temperature for 45 minutes. TLC showed the reaction complete. The solid was filtrated off and the residue was washed by anhydrous acetonitrile. The solvent was removed under reduced pressure. The residue was fractioned by silica gel chromatography with anhydrous ethyl acetate: hexane 3:1. A light yellow solid was obtained by precipitation with anhydrous hexane from trace amount of anhydrous ethyl acetate. 12.4mg, 94.6%. MS: C₃₃H₄₀N₂O₁₂, cal. 657.2659 (M+H⁺), found 657.2669. This compound is 3b, see FIG. 4.

### Example 5

### Synthesis of WmC20-Proline

Wortmannin (10.7 mg, 0.025 mmol) and proline (5.75 mg, 0.05 mmol) were mixed in anhydrous DMSO (0.5 mL). The mixture was stirred at room temperature for 1 hr. After dilution with 50% acetonitrile in water (1:1), the mixture was purified by HPLC (system 1) and gave a yellow powder after lyophilization. Analysis of 1 by HPLCshowed 89.5% in purity. Yield: 13.5 mg, 99.5%. MS: C₂₈H₃₃NO₁₀, cal. 544.2182, found 544.2179 (M+H⁺); ¹H NMR, (CDCl₃): 0.86 (3H, s, 13-CH₃), 1.31-1.35 (1H, t, J=8 Hz, H-12), 1.47 (3H, s, 10-CH₃), 1.70-2.00 (5H, b, 20H, CH₂CH₂CH₂CHCOOH, H-15), 2.15 (3H, s, CH₃COO), 2.20-2.35 (2H, m, H1-16, NCH₂), 2.35-2.45 (1H, m, NCH₂), 2.55-2.64 (2H, m, H-12, H-16), 2.90-3.00 (2H, m, H-14, CH₃OCH₂), 3.10-3.20 (3H, m, H-15, NCH₂CH₂CH₂CHCOOH), 3.25 (3H, s, CH₃OCH₂), 3.40-3.50 (1H, b, MeOCH₂), 4.50-4.65(2H, m, H-1, NCHCOOH), 6.00-6.10 (1H, b, H-11), 8.28 (1H, s, H-20).

### Example 6

### Synthesis of WmC20NH-N(Ac)lysine derivative

Wortmannin (12.8 mg, 0.03 mmol), N-acetyl lysine (28.2 mg, 0.15 mmol), and triethylamine (30 μL) were mixed in a mixture of DMSO (1 mL), methanol (1 mL) and water 100 μL. After 1 hr stirring, the reaction mixture was purified by reverse C18 HPLC, and a yellow powder was obtained after lyophilization. Yield: 14.5 mg, 78.4%. MS: C₃₁H₄₀N₂O₁₁, cal. 617.2710, found 617.2739 (M+H⁺); NMR (CDCl₃): 0.81 (3H, s, 13-CH₃), 1.38-1.60 (2H, m, —NHCH₂CH₂CH₂CH₂CH—), 1.52 (3H, s, 10-CH₃), 1.64-1.76 (2H, m, —CH₂CH₂CH₂CH₂CH—), 1.82-1.96 (3H, H-12, NHCH₂CH₂CH₂CH₂CH—), 2.04 (3H, s, NHCOCH₃), 2.06 (3H, s, CH₃COO), 2.22-2.40 (3H, m, H-15, 2H-16), 2.50-2.63 (1H, m, H-12), 2.80-2.99 (2H, m, H-14, CH₃OCH₂), 3.16-3.24 (2H, m, H-15, CH₃OCH₂), 3.26 (3H, s, CH₃OCH₂), 3.36-3.52 (2H, m, NHCH₂), 3.52-3.89 (2H, OH), 4.30-4.35 (1H, m, H-1), 4.70-4.78 (1H, m, CH₂CHCOOH), 5.97-6.01 (1H, m, H-11), 6.94 (1H, d, J=8 Hz, NHCOCH₃), 8.55 (1H, d, J=12 Hz, H-20), 9.83-9.88 (1H, m, C20NH).

### Example 7

### Synthesis of WmC20NAc-Cys

Wortmannin (17.1 mg, 0.04 mmol), N-acetyl cysteine (13 mg, 0.08 mmol), and triethylamine (0.4 mL) were mixed in CH2C12 (1 mL). After 1.5 hr stirring, the reaction mixture was concentrated under reduced pressure. The residue was purified by HPLC (system 3), and a yellow powder was obtained after lyophilization. Subsequent purity analysis by reverse phase C18 HPLC revealed the presence of a minor impurity, accounting for less than 20% of total. Yield: 19.4 mg, 81.8%. MS: C₂₈H₃₃N₂O₁₁S, cal. 592.1852, found 592.1856 (M+H⁺), 609 (M+NH₄⁺); 1H NMR, (CDCl₃): 0.82 (3H, s, 13-CH₃), 1.52 (3H, S, 10-CH₃), 1.70-2.01 (3H, b, H-12, 2OH), 2.05 (3H, s, NHCOCH₃), 2.09 (3H, s, CH₃COO), 2.22-2.40 (3H, m, H-15, H-16), 2.54-2.64 (1H, m, H-12), 2.80-2.90 (1H, m, H-14), 2.94-3.00 (1H, m, CH₃OCH₂), 3.14-3.34 (2H, m, H-15, CH₃OCH₂), 3.26 (3H, S, CH₃OCH₂), 3.40-3.50 (2H,AB, J1=4 Hz, J2=16 Hz, SCH₂), 4.46 (1H, d, J=4 Hz, H-1), 4.98-5.00 (1H, m, CHCOOH), 5.98-6.01 (1H, m, H-11), 6.59 (1H, d, J=8Hz, NHCOCH₃), 8.78 (1H, s, H-20).

### Example 8

### Synthesis of WmC20NH-hexane diamine

Wortmannin (8.6 mg, 0.02 mmol), Boc-mono-protected 1,6 diaminohexane (8.8 mg, 0.04 mmol), and triethylamine (10 μL) are mixed in CH₂Cl₂ (1 mL). After 1.5 hr stirring, the reaction mixture is concentrated under reduced pressure. The residue is purified by silica gel eluted with hexane and ethyl acetate. The crude product is dissolved in CH₂Cl₂ with 20% of TFA. The deprotection is under the room temperature stirring for 1 hr. The solvent is evaporated under reduced pressure. The crude product was purified by silica gel column chromatography. The amine group of the linker can form amide bonds with carboxylic acid bearing targeting groups.

### Example 9

### Synthesis of WmC20NH-ethylene diamine

Wortmannin (8.6 mg, 0.02 mmol), Boc-mono-protected ethylenediamine (6.4 mg, 0.04 mmol), and triethylamine (10 μL) are mixed in CH₂Cl₂ (1 mL). After 1.5 hr stirring, the reaction mixture is concentrated under reduced pressure. The residue is purified by silica gel eluted with hexane and ethyl acetate. The crude product is dissolved in CH₂Cl₂ with 20% of TFA. The deprotection is under the room temperature stirring for 1 hr. The solvent is evaporated under reduced pressure. The crude product was purified by silica gel column chromatography.

### Example 10

### Synthesis of WmC20NH-aminoethanol

Wortmannin (8.6 mg, 0.02 mmol), ethanolamine (3 μL, 0.049 mmol), and triethylamine (10 μL) are mixed in CH₂Cl₂ (1 mL). After 1 hr stirring, the reaction mixture is concentrated under reduced pressure. The residue is purified by silica gel chromatography eluted with hexane and ethyl acetate. Hydroxyl group of the linker can be reacted with carboxylic acids, to yield ester linked targeting groups.

### Example 11

### Synthesis of WmC20-S-propionate

To a solution of a mixture of 3-mercaptoprionic acid (18 μL, 0.2 mmol) and wortmannin (8.65 mg, 0.02 mmol) in chloroform (0.5 mL) was added triethyl amine (14 μL, 0.1 mmol). After stirring for 24 hours at room temperature, the mixture is fractioned via SiO₂ chromatography. A yellow powder can be obtained by precipitating from the solution of dichloromethane by hexane addition The carboxy function on the WmC20 derivative can be converted to an NHS ester using carbodiimide and N-hydroxysuccinmide as in examples 3 and 4.

### Example 12

### Synthesis of a wortmannin Wm-N(Me)Hexanoate-BSA conjugate

BSA (25 mg) was dissolved in 1 mM PBS buffer at pH 6.8 (500 μL) and a solution of 3a (2.0 mg, 0.003 mmol) in DMSO (50 μL) was added. The mixture was vortexed for a while and the solution became clear. It was incubated at 37° C. for 1 hour. The solution was fractioned by PD-10 column eluted by 1 mM PBS buffer. The fraction was collected and detected by UV absorbance at 280 nm. The ratio of BSA to 2a was calculated by dividing the moles of 2a with the moles of BSA. The moles of BSA was calculated based on the UV absorbance at 280 nm by the standard curve after the subtraction of the absorbance of 2a from the total at 280 nm. The moles of wortmannin were calculated based on the absorbance of 418 nm by the standard curves of the concentrations of 2a versus the absorbance at 418 nm. The ratio is BSA:2a=1:4.9. Coupling efficiency: 98%.

### Example 13

### Synthesis of a WmC20-NH-hexanoate-BSA conjugate

BSA (50 mg) was dissolved in 1 mM PBS buffer at pH 6.8 (1 mL) and a solution of 2b (4.0 mg, 0.006 mmol) in DMSO (120 μL) was added. The mixture was vortexed for a while and a clear solution was got after it was incubated at 37° C. for 1 hour. The solution was fractioned by PD-10 column eluted by 1 mM PBS buffer. The fraction was collected and detected by UV absorbance at 280 nm. The ratio of BSA to 2b was calculated by dividing the moles of 2b with the moles of BSA. The moles of BSA was calculated based on the absorbance of 280 nm by correlating to the standard curve after the subtraction of the absorbance of 2b from the total. The moles of wortmannin was calculated based on the absorbance of 408 nm by the standard curves of the concentrations of 2b versus the absorbance at 408 nm. The ratio is BSA:2b-1:8.7.

### Example 14

### Synthesis of WmC20-N(Me)-hexanoate-IgG

Mouse IgG (5 mg) was dissolved in 1 mM PBS buffer at pH 6.8 (1 mL) and a solution of 3a (0.86 mg, 0.0012 mmol) in acetonitrile (30 μL) was added. The mixture was votexed and incubated at 37° C. for 1.5 hours. The solution was fractioned by PD-10 column eluted by 1 mM PBS buffer. The fraction was collected and detected by TV absorbance at 280 nm. The ratio of IgG to 2a was calculated by dividing the moles of 2a with the moles of IgG. The moles of IgG was calculated based on the absorbance of 280 nm by the standard curve after the subtraction of the absorbance of 2a from the total at 280 nm. The moles of wortmannin were calculated based on the absorbance of 418 nm by the standard curves of the concentrations of 2a versus the absorbance at 418 nm.

The ratio is IgG:2a=1:23. Coupling efficiency: 57.5% based on 3a. Variations of this method can be employed monoclonal antibodies including monoclonals now used clinically.

### Example 15

### Synthesis of WmC20-NH-hexanoate-IgG

Mouse IgG (4 mg) was dissolved in 1 PBS buffer at pH 6.8 (1 mL) and a solution of 3b (1.03 mg, 0.0016 mmol) in acetonitrile (80 μL) was added. The mixture was vortexed and incubated at 37° C. for 1.5 hours. The solution was fractioned by PD-10 column eluted by 1 mM PBS buffer. The fraction was collected and detected by UV absorbance at 280 nm. The ratio of IgG to 2b was calculated by dividing the moles of 2b with the moles of IgG. The moles of IgG were calculated based on the absorbance of 280 nm by correlating to the standard curve after the subtraction of the absorbance of 2b from the total. The moles of wortmannin was calculated based on the absorbance of 408 nm by the standard curves of the concentrations of 2b versus the absorbance at 408 nm. The ratio is IgG:2b=1:31.5. Coupling efficiency: 52% based on 3b. Variations of this method can be employed monoclonal antibodies including monoclonals now used clinically.

### Example 16

### Synthesis of WmC2O-N(Me)-hexanoate-Bead

Dynal M-270 amine magnetic beads 600 μL with 1.2×10⁹ beads and 2.93 μmol amine functionality (30 mg/mL, 150-175 μmol/g Dynal beads) were washed by PBS pH 6.8 three times (3×1 mL). The beads were suspended in a mixture of DMSO and PBS buffer (pH 6.8) (1:1, V/V) (500 μL). One equivalent 3a in anhydrous DMSO was added and the mixture was incubated at 37° C. for 1 hour while the tube was shaken for a few times. Then the beads were washed with PBS buffer (H 6.8) (5×1 mL) with the assistance of a magnet. The beads (6a) were stored at −80° C.

### Example 17

### Synthesis of WmC20-N(Me)-hexanoate-dextran

Amino dextran (MW: 40,000) (20 mg, 0.0005 mmol) was dissolved in PBS (pH=6.8) (0.3 mL). (3a) (6.7 mg, 0.01 mmol) in acetonitrle (0.05 mL) was transferred into the dextran solution above. DMSO (0.2 mL) was added to prevent the precipitation of 3a. The mixture was incubated under 37° C. for 1.5 hours. The product was purified by the method of precipitation with acetonitrile (45 mL). The solid was collected by spinning with a centrifuge at high speed. This process was repeated for three times. A dried sample was obtained by lyopholization. The coupling ratio between 2a and dextran is 2a/dextran=10.5 which was calculated by the UV standard curve of 2a at 418 nm

### Example 18

### Synthesis of WmC20-N(H)-hexanoate-dextran.

Amino dextran (MW: 40,000) (20 mg, 0.0005 mmol) was dissolved in PBS (pH=6.8) (0.3 mL). WmC20NHHA-NHS (3b) (6.6 mg, 0.0115 mmol) in acetonitrile (0.084 mL) was transferred into the dextran solution above. DMSO (0.2 mL) was added to prevent the precipitation of 3b. The mixture was incubated under 37° C. for 1.5 hours. The product was purified by PD-10 column eluted by 1 mM phosphate buffer at pH 7.0. The sample was dried via lyopholization. The coupling ratio between 2b and dextran is 2b/dextran-9.3 which was calculated by the UV standard curve of 2b at 408 nm.

### Example 19

### Synthesis of WmC20-proteins through attachment of lysine

A general method of synthesizing WmC20 conjugates with a secondary amine at C20 is by reaction the epsilon amino groups of proteins. In case the linker is a bond between the lysine amine nitrogen atom and the C20 position of the wortmannin-like compound. One or more than one lysine side chains can be modified by wortmannin-like compound by adjusting the relative amounts of protein and wortmannin-like compound. A problem with this method is, however, the limited solubility of wortmannin in aqueous media and the limited toleration of proteins for organic solvents. A method of overcoming these solubility limitation is to synthesize an WmC20N(Me)-R derivative where R is a hydrophilic group that improves the water solubility of the wortmannin-like compound. This soluble WmC20 derivative is then taken into an aqueous solution containing the desired protein targeting group to form the desired WmC20 protein conjugate as provided in the following description.

WmC20-N-methyl-glucamine was prepared by combining wortmannin (21.4 mg, 0.05 mmol) and N-methyl-glucamine (19.5 mg, 0.1 mmol) in anhydrous DMSO (0.5 mL) at room temperature with stirring for 0.5 hours. The product was purified by HPLC and lyophilized. Yield 30.7 mg, 98.7%.

A BSA wortmannin conjugate can be prepared by combining WmC20-N-methyl-glucamine (2.4 mg, 3.79×10⁻³ mmol) with BSA (25 mg, 3.79×10⁻⁴ mmol) in phosphate buffered saline (0.4 mL) at 37° C. with stirring overnight. The mixture can be purified by size exclusion chromatography. The ratio of Wm to BSA in the product can be calculated using a standard curve.

### Example 20

### Synthesis of WmC20-N(Me)-hexanoate-transferrin

Human holo-transferrin (40 mg, 0.5 μmol) was dissolved in PBS (pH 7.0, 0.8 mL)-3a (example 3, 2 mg, 2.98 μmol) in acetonitrile (20 μL) was added. The mixture was incubated under 37° C. for 1.5 hours. The mixture was fractioned by a column of Sephadex G-50 eluted by 1 mM phosphate buffer at pH 7.0. A brown powder was obtained after lyophilization. The coupling ratio between 2a/transferrin (2.88) was calculated by a standard curve of 2a at 418 nm.

### Example 21

### Synthesis of WmC20-N(H)-hexanoate-tansferrin

Human holo-transferrin (40 mg, 0.5 μmol) was dissolved in PBS (pH 7.0, 0.8 mL). 3b (example 4, 1.7 mg, 2.5 μmol) in acetonitrile (20 μL) was added. The mixture was incubated under 37° C. for 2 hours. The mixture was fractioned by a column of Sephadex G-50 eluted by 1 mM phosphate buffer at pH 7.0. A brown powder was obtained after lyophilization. The coupling ratio between 2b/transferrin (2.77) was calculated by a standard curve of 2b at 408 nm.

### Example 22

### Synthesis of WmC20-Proline-bombesin peptide

A general method for synthesizing WmC20 conjugates with a tertiary amine at C20 is to react Wm with a peptide which has an N-terminal proline as linker. In this case the linker (L) is an N terminal proline and the targeting moiety (T) consists of all other amino acids so that the general structure is WmC20-Pro-Peptide. Thus the linker and targeting moiety are combined before reaction with Wm. To a clear solution of wortmannin (4.28 mg, 10 mol) and peptide ([H]-PGDDGQWAVGHLM-[NH2]) (13.8 mg, 10 μmol) in DMSO, was added triethylamine (20 μL). The reaction was complete in 15 minutes, which was indicated by the disappearance of wortmannin by TLC. The mixture was diluted by a solution of acetonitrile and water (1:1, v/v) in 1:1 ratio. The conjugate can be obtained by HPLC purification eluted by acetonitrile and water in a program of gradient. The product was identified by MS: 1811 (M+1).

### Example 23

### Synthesis of WmC20-N(H)-hexanoate-Bombesin Peptide

To a solution of peptide ([Ac]-KEEEQWAVGHLM-[NH2]) (3.8 mg, 5 μmol) and 2b-NHS ester (3.48 mg, 5 μmol in anhydrous acetonitrile 40 μL) in anhydrous DMSO (0.5 mL), was added triethyl amine (10 μL). The mixture was incubated at 37-40° C. for 1 hour, then the solution was diluted by a mixture of acetonitrile and water (1/1, v/v) in 1:1 ratio. The product was purified by HPLC and analyzed by MS of 2040 (M+1).

### Example 24

### Synthesis of a Wortmannin-C20N(Me)-Folate Conjugate

An amine group was attached to folic acid as described by Moon et al., Bioconjugate Chem. 14:539 (2003). A mixture of folic acid (0.238 g, 0.5 mmol) and diisopropylcarbodiimide (DIC, 0.088 g, 0.5 mmol) in anhydrous DMSO (5 mL) was stirred at room temperature for 1 hour. To this solution was added 1,8-bis(methylamino)-3,6-dioxaoctane (0.77 mL, 5 mmol). After stirring for 5 hours at room temperature, water (5 mL) was added and the solid precipitated was collected by centrifuge. The solid was redissolved in DMSO and purified by reverse phase HPLC with C4 column. A yellow powder was obtained after lyophilization which was identified by MS: C₂₇H₃₇N₉O₇: Cal. 599, found: 600 (M+1). The purified intermediate, folate amine, reacted with wortmannin. The resulting wortmannin conjugate was isolated by RP-HPLC. MS: cal C₅₀H₆₁N₉O₁₅, Ca;=1028.4365 (M+1), Found 1028.4393 (M+1). Folate can interact with the folate receptor and target the Wm conjugate.

### Example 25

### Synthesis of WmC20-cetuximab

A mixture of wortmannin (21.4 mg, 0.05 mmol) and N-methyl glucamine (19.5 mg, 0.1 mmol) in anhydrous DMSO (0.5 mL) was stirred at room temperature for 0.5 hours. The mixture was purified by HPLC. A yellow powder, termed WmC20N(Me) glucamine, was obtained after lyophilization. Yield, 30.7 mg, 98.7%. A solution of Erbituxam (aka cetuximab, 30 mg) and WmC20N(Me)glucamine (1.36 mg, 2.18×10⁻³ mmol) were (combined in PBS (5 mL) at 37° C. for 20 hours. Unreacted WmC20N(Me)glucamine was removed with a Sephadex G-50 column in 1 mM phosphate buffer at pH 7. After lyophilization, a yellow powder was obtained. The ratio of Wm to cetuximab (6.0 Wm/mole) was obtained from the absorbances of certuximab at 280 nm and conjugated Wm at 408 nm. The coupling efficiency was 55%. The WmC20-centuximab can be checked for immunoreactivity by using A549 cells and erbitux.

### Example 26

### Synthesis of WmC20-trastuzumab

WmC20N(Me)glucamine is prepared as described in example 25 and reacted with trastuzumab as described in example 25. Trastuzumab (Herceptin) is dialyzed to remove amine containing histidine.

### Example 27

### Synthesis of WmC20-abciximab

WmC20N(Me)glucamine is prepared as described in example 25 and reacted with abciximab (Reopro) as described in example 25.

### Example 28

### Synthesis of WmC20 NH-glycine-glucosamine

Wortmannin (8.6 mg, 0.02 mmol), t-butyl glycine ester hydrochloride (6.7 mg, 0.04 mmol), and triethylamine (20 μL) is mixed in anhydrous acetonitrile (1 mL). The mixture is stirred at room temperature until the reaction is complete as shown by TLC. The final solution is purified by silica gel chromatography.

WmC20NH-glycine-tert-butyl ester (55.9 mg, 0.1 mmol) is dissolved in a solution of 50% TFA in anhydrous methylene chloride (2 mL) to deprotect. the carboxylate group. The solution is stirred under ambient temperature for couple-of hours. The residue after the evaporation of solvent is purified by silica gel column chromatography. WmC20NH-glycine is obtained WmC20NH-glycine (50.3 mg, 0.1 mmol), DCC and N-hydroxysuccinimide are mixed in acetonitrile and stirred at ambient temperature for a few hours. An NHS ester of WmC20NH-glycine is obtained after the purification of the crude product by silica gel preparative TLC plate.

Compound 28 is made by the reaction of WmC20NH-glycine NHS ester with glucosamine in DMSO at ambient temperature. The final compound is obtained by HPLC purification.

### Example 29

### Synthesis of WmN(Me) glycine glucosamine

Wortmannin (8.6 mg, 0.02 mmol), sarcosine (3.6 mg, 0.04 mmol), and triethylamine (20 μt) are mixed in anhydrous acetonitrile (1 mL). The mixture is stirred at room temperature until the reaction is complete as shown by TLC. The final solution is purified by silica gel chromatography

WmC20-sarcosine (51.7 mg, 0.1 mmol), DCC and N-hydroxysuccinimide are mixed in acetonitrile and stirred at ambient temperature for a few hours. An NHS ester of WmC20NMe-glycine is obtained after the purification of the crude product by silica gel preparative TLC plate.

Compound 29 is made by the reaction of WmC20sarcosine NHS ester with glucosamine in DMSO at ambient temperature. The final compound is obtained by HPLC purification.

### Example 30

### Synthesis of WmC20N(Me)-Mtx

A similar procedure as example 24 is used to make compound 31. An amine group is attached to folic acid as described by Moon et al., Bioconjugate Chem. 14:539 (2003). Briefly, methotrexate is reacted with 2,2-(ethylenedioxy)bis(N-methylethylamine) using diisopropylcarbodiimide as the coupling agent in DMSO. The resulting compounds are purified by RP-HPLC and reacted with the wortmannin. The resulting wortmannin conjugate is isolated by RP-HPLC. Methotrexate conjugates can be used to enhance the potency of a wortmannin-like compound.

### Example 31

### Synthesis of WmC20NH-Mtx

A similar procedure as example 24 is used to make compound 30. An amine group is attached to folic acid as described by Moon et al., Bioconjugate Chem. 14:539 (2003). Briefly, methotrexate is reacted with 2,2-(ethylenedioxy)bis(ethylamine) using diisopropylcarbodiimide as the coupling agent in DMSO. The resulting compounds are purified by RP-HPLC and are reacted with the wortmannin. The resulting wortmannin conjugate is isolated by RP-HPLC. Methotrexate is used to enhance the potency of a wortmannin-like compound.

### Example 32

### Synthesis of WmC20NH-Paclitaxel

(−)-Paclitaxel-2′-hemisuccinate (95.3 mg, 0.1 mmol) and N-Doc-2,2′-(ethylenedioxy)diethylamine (25 mg, 0.2 mmol) are mixed together with DIC in methylene chloride (2 mL). A product of (−)-Paclitaxel-2′-hemisuccinate tethered with N-Boc-2,2′-(ethylenedioxy)diethylamine is isolated by silica gel chromatography. The Boc protected amino group is released by 50% TFA deprotection in methylene chloride. This free amino group is used to react directly with C20 position of wortmannin to make WmC20 NH-paclitaxel conjugate (32).

### Example 33

### Synthesis of WmC20N(Me)-Paclitaxel

(−)-Paclitaxel-2′-hemisuccinate (95.3 mg, 0.1 mmol) and 1,8-Bis(methylamino)-3,6-dioxaoctane(0.176 g, 1 mmol) are mixed together with DIC in methylene chloride (2 mL). A product of (−)-Paclitaxel-2′-hemisuccinate tethered with 1,8-Bis(methylamino)-3,6-dioxaoctane is isolated by HPLC. This free amino group is used to react directly with C20 position of wortmannin to make WmC20 NMe-paclitaxel conjugate (33).

### Example 34

### Stability of Wortmannin C20 Derivatives

The stability of compounds of the invention was obtained by the following method. The decay of wortmannin in the presence of N-acetyl lysine and proline was determined by analyzing the solution of wortmannin (about 1.5 μM) of PBS buffer with 10 mM of N-acetyl lysine and proline (pH 6.8, 400 μL) and acetonitrile (60 μL) in a 1.5 mL tubes. The solutions were incubated at 37° C. At different time point, 10 μL solution was mixed with 1 μL internal standard solution in DMSO (containing 1 μg methyl-4-hydroxybenzoate) and analyzed by HPLC. The initial time point (0 hr) was taken before putting the tube in the water bath. Before the injection, the samples were vortexed briefly and then centrifuged at room temperature.

The decay of wortmannin in the presence of BSA was determined by mixing wortmannin (0.45 mg, 1.04 μmol) and BSA (1.8 mg) in the mixture of PBS buffer (pH 6.8, 400 μL) and acetonitrile (60 μL) in a 1.5 mL tubes. The solutions were incubated at 37° C., At different time point, 10 μL solution was mixed with 1 μL internal standard solution in DMSO (containing 1 μg methyl 4-hydroxybenzoate, MHB) and analyzed by HPLC. The initial time point (0 hr) was taken before putting the tube in the water bath. Before the injection, the samples were vortexed briefly and then centrifuged at room temperature.

Stability data was fit to equation 1, which illustrates our treatment of the data for 2a (initial concentration=[2a]ₜ, concentration as a function of time=[2a]ᵢ) as shown in FIG. 2 with first order decay constants (k_{FOD}) given in Table 1.

[2a]ₜ=[2a]ᵢ exp(−k_{FOD}t) Eq 1:

TABLE 1
First Order Decay Constants for Wortmannin and Wortmannin
Derivatives

| | | K_{FOD} (hr⁻¹) | | |
|---|---|---|---|---|
| | | (95% | | |
| Compound | Conditions | confidence intervals) | Half Life | |
| Wm | PBS | 0.012 (0.0080-0.016) | 57.8 | hr |
| Wm | PBS, 0.065 mM | 0.18 (0.16-0.20) | 3.85 | hr |
| | BSA | | | |
| Wm | PBS, 10 mM | 1.87 (0.62-3.13) | 22 | min |
| | Proline | | | |
| Wm | PBS, 10 mM | 0.30 (0.23-0.38) | 2.31 | hr |
| | NAcLys | | | |
| FIG. 4, 2a | PBS | 0.080 (0.071-0.089) | 8.7 | hr |
| FIG. 4, 2a | PBS, 10 mM | 0.065 (0.0062-0.068) | 10.7 | hr |
| | Proline | | | |
| FIG. 4, 2b | PBS | 0.0053 (0.0040-0.0067) | 131 | hr |
| FIG. 4, 2b | PBS, 10 mM | 0.0031 (0.0023-0.0038) | 223 | hr |
| | Proline | | | |
| Wm | RPM* | 4.02 | 10.3 | min |
| Wm | MEM* | 5.04 | 8.3 | min |

*Data from Holleran et al., Anal. Biochem. 323:19 (2003).

### Example 35

### Formation of wortmannin from WmC20 conjugates monitored by HPLC

The solutions of 2a, 2b, or wortmannin (about 1.5 mM, 200 μL) were made by reconstituting the 30 mM stock solution in DMSO with PBS buffer (pH 6.8). The solutions were incubated in water bath at 37° C. and were analyzed at different time points with HPLC by injecting the solution (10 μL) together with 1 μL internal standard solution (containing 1 μg methyl 4-hydroxybezoate). The initial time point (0 hr) was taken before putting the tube in the water bath. HPLC gradients were adjusted to produce baseline separations. Before the injection, the samples were vortexed briefly and then spun down. Data using this method appear in FIG. 6.

Compounds (1.8 mg) and 4b (1.3 mg) (examples were weighted into the 1.5 mL tubes and dissolved in PBS buffer (pH 6.8, 400 μL). The solutions were incubated at 37° C. At different time point 20 μL solution was mixed with 1 μL internal standard solution in DMSO (containing 1 μg methyl 4-hydroxybenzoate) and analyzed by HPLC. The initial time point (0 hr) was taken before putting the tube in the water bath. Before the injection, the samples were vortexed briefly and then spun down.

HPLC chromatograms demonstrating wortmannin formation are shown in FIG. 6; FIG. 6C shows the formation of wortmannin for the WmC20 conjugate of example 12. The appearance of wortmannin was modeled according to equation 2 which is based on the model of FIG. 7A.

[Wortmannin]ₜ=[2a]ᵢk₁[exp(−k₁t)−exp(−k₂t)]/(k₁−k₂) Eq 2:

Equation 2 (model FIG. 7A) was fit to HPLC data obtained with WmC20conjugates from examples 12, 14 and 16. Data is summarized in Table 2.

TABLE 2
Constants for Wortmannin Generation from Wortmannin Derivatives
(Fit to Equation 2)

| | Initial | | |
|---|---|---|---|
| | Concentration | | |
| | Of Wm | | |
| | Derivative, | | |
| Compound | (as mM Wm) | K₁, (hr⁻¹) | K₂, (hr⁻¹) |
| Example 1 | 0.94 | 0.051 (0.045-0.056) | 0.114 (0.095-0.132) |
| Example 12 | 0.35 | 0.190 (0.128-0.253) | 0.905 (0.707-1.103) |
| Example 14 | 0.35 | 0.024 (0.013-0.034) | 0.282 (0.166-0.398) |
| Example 16 | 0.0750 | 0.308 (0.124-0.492) | 0.148 (0.076-0.220) |
| | (0.0414-0.109) | | |

Concentrations of BSA and IgG were 0.065 mM and 0.011 mM, respectively.
Concentration of beads was 3 × 10⁹ bead/mL (7.3 umol/mL functionality).

### Example 36

### Formation of wortmannin from WmC20 conjugates monitored by the inhibition of PI3K enzyme activity

The generation of wortmannin from WmC20 derivatives and conjugates was obtained by HPLC was confirmed by measuring the inhibition of PI3K enzyme activity. We incubated compounds for three hours at 37° C. in PBS, pH 6.8, followed a 30 minute incubation for PI3K activity using the assay system previously described which involves lipid phosphorylation by ³²P labeled ATP and thin layer chromatography, (see Yuan et al., Bioconjug. Chem. 16:669 (2005)). IC50's are given below in Table 3. Also shown are the percentages of wortmannin released from the WmC20 derivative after three hours in PBS as obtained using HPLC.

TABLE 3
Inhibition of PI3K by Wortmannin Derivatives and Wortmannin
Conjugates

| | | | | Wm |
|---|---|---|---|---|
| | Bioassay: PI3K IC50 (nM) | | IC50Wm/ | Generation |
| | (95% confidence | | IC50 Wm- | By HPLC |
| Compound | intervals) | | derivative | (3 hr, 37° C.) |
| wortmannin | 4.3 ± 1.3 | (2.5-7.4) | NA | NA |
| Example 1 | 36.7 ± 1.0 | (34.5-39.0) | 0.117 | 10.4% |
| Example 2 | >1000 | | <0.004 | ND |
| Example 12 | 14.8 ± 1.1 | (12.6-17.4) | 0.344 | 16.5% |
| Example 13 | >1000 | | <0.004 | ND |
| Example 14 | 129.9 | (104.4 to 161.5) | 0.033 | 3.5% |

| | | | |
|---|---|---|---|
| Example 15 | >1000 | <0.004 | ND |

### Example 37

### Anti-proliferative assay for WmC20 conjugates

A549 cells were grown in RPMI-1640 supplemented with 10% FBS, 100 units/mL of penicillin, 100 μg/mL of streptomycin, and 2 mM L-glutamine. All cells were grown in a humidified atmosphere at 37° C. and 5% CO₂. Growth inhibition was evaluated by the SRB assay²². Cells were seeded in 96-well plates at 5000 cells per well in 100 μL of media and incubated at 37° C. for 24 hours. Cells were then treated with 0-100 μM of compounds at 37° C. for 48 hours. Cells were fixed with 50 μL cold 50% TCA, incubated at 4° C. for 1 hour, and washed 5 times with tap water. The 96-well plates were allowed to air dry and 100 μL 0.4% sulforhodamine B (SRB) in 1% acetic acid was added to each well. After 10 minutes, the unbound SRB was washed away with 1% acetic acid and the plates were dried. Protein-bound dye was solubilized by addition of 200 μL 10 mM Tris buffer and subsequent shaking at RT for 10 minutes. The absorbance of each well was measured at 490 nm by a plate reader. Percent cell survival was calculated for each concentration by the ratio of the measured absorbance to the absorbance of the untreated cells. Curve fitting was performed using Prism 4.0a software. IC50 is the 50% inhibition of cell mass without subtraction of the resistant cell population.

Data are shown in Table 4.

TABLE 4
Anti-proliferative Activity of Low Molecular Weight WmC20
Derivatives and High Molecular Weight WmC20 Conjugates

| | IC50 (μM) | IC50 (μM) |
|---|---|---|
| Compound | A549 | HeLa |
| wortmannin | 21.1 ± 0.5 | 41.0 ± 0.9 |
| Example 1 | 3.11 ± 0.17 | 10.9 ± 0.2 |
| Example 2 | 45.8 ± 0.3 | 63.8 ± 0.6 |
| Example 17 | 3.96 ± 0.23 | 13.3 ± 0.4 |
| Example 18 | 64.7 ± 1.2 | 58.0 ± 0.6 |

### Example 38

### HPLC Methods

The following HPLC methods were employed. HPLC (Varian Prostar 210 with a variable wavelength PDA 330 detector) employed reverse phase C18 columns (VYDAC, Cat.#: 218TP 1022 for synthesis; Varian, Cat. #: R0086200C5 for analysis) with water (Millipore, containing 0.1% trifloroacetic acid,) (buffer A) and acetonitrile (containing 20% buffer A) (buffer B) as elution buffer. System 1: buffer A/buffer B (80:20) isocratic for 5 minutes, linear gradient to buffer A/buffer B (20:80) over 40 minutes, then the gradient back to 80:20 in 5 minutes and isocratic for 5 minutes, flow: 4.9 ml/min, λmax: 418 nm (used for purification of 2a); System 2: buffer A/buffer B (80:20) isocratic for 5 minutes, linear gradient to buffer A (buffer B (20:80) over 25 minutes and then isocratic for 5 minutes, then the gradient back to 80:20 in 5 minutes and isocratic for 5 minutes, flow: 6.0 mi/min, λmax: 408 nm (used for purification of 2b); System 3: buffer A/buffer B (70:30) linear gradient to buffer A/buffer B (20:80) over 15 minutes, then gradient back to 70:30 in 3 minutes and isocratic for another 5 minutes, flow: 1.0 ml/min, λmax: 258 nm (System 4: buffer A/buffer B (99:1) linear gradient to buffer A/buffer B (40:60) over 46 minutes, then gradient back to 90:1 in 5 minutes and isocratic for another 5 minutes, flow: 1.0 ml/min, λmax: 250 nm).

### Example 39

### Synthetic Methods for the synthesis of L−T

The linking of a wortmannin-like compound to a targeting group is achieved by covalent means, involving bond formation at C20 of the wortmannin-like compound with a nitrogen atom or sulfur atom located on a linker or a targeting group. Where the linker provides a molecular skeleton for covalently linking the wortmannin derivative to a targeting group, a variety of functional groups can be employed to form a covalent bond with the targeting group. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

For example, a hydroxyl group of the targeting group may react with a carboxyl group of the linker, or an activated derivative thereof, resulting in the formation of an ester linking the two.

Examples of moieties capable of reaction with sulfhydryl groups include α-haloacetyl compounds of the type XCH₂CO— (where X═Br, Cl or I), which show particular reactivity for sulfhydryl groups, but which can also be used to modify imidazolyl, thioether, phenol, and amino groups as described by Gurd, Methods Enzymol. 11:532, 1967. N-Maleimide derivatives are also considered selective towards sulfhydryl groups, but may additionally be useful in coupling to amino groups under certain conditions. Reagents such as 2-iminothiolane (Traut et al., Biochemistry 12:3266, 1973), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:
(i) α-haloacetyl compounds, which show specificity towards amino groups in the absence of reactive thiol groups and are of the type XCH₂CO—(where X═Cl, Br or I), for example, as described by Wong Biochemistry 24:5337, 1979;
(ii) N-maleimide derivatives, which may react with amino groups either through a Michael type reaction or through acylation by addition to the ring carbonyl group, for example, as described by Smyth et al., J. Am. Chem. Soc. 82:4600, 1960 and Biochem. J. 91:589, 1964;
(iii) aryl halides such as reactive nitrohaloaromatic compounds;
(iv) alkyl halides, as described, for example, by McKenzie et al., J. Protein Chem. 7:581, 1988;
(v) aldehydes and ketones capable of Schiff's base formation with amino groups, the adducts formed usually being stabilized through reduction to give a stable amine;
(vi) epoxide derivatives such as epichlorohydrin and bisoxiranes, which may react with amino, sulfhydryl, or phenolic hydroxyl groups;
(vii) chlorine-containing derivatives of s-triazines, which are very reactive towards nucleophiles such as amino, sufhydryl, and hydroxyl groups;
(viii) aziridines based on s-triazine compounds detailed above, e.g., as described by Ross, J. Adv. Cancer Res. 2:1, 1954, which react with nucleophiles such as amino groups by ring opening;
(ix) squaric acid diethyl esters as described by Tietze, Chem. Ber. 124:1215, 1991; and
(x) α-haloalkyl ethers, which are more reactive alkylating agents than normal alkyl halides because of the activation caused by the ether oxygen atom, as described by Benneche et al., Eur. J. Med. Chem. 28:463, 1993.

Representative amino-reactive acylating agents include:
(i) isocyanates and isothiocyanates, particularly aromatic derivatives, which form stable urea and thiourea derivatives respectively;
(ii) sulfonyl chlorides, which have been described by Herzig et al., Biopolymers 2:349, 1964;
(iii) acid halides;
(iv) active esters such as nitrophenylesters or N-hydroxysuccinimidyl esters;
(v) acid anhydrides such as mixed, symmetrical, or N-carboxyanhydrides;
(vi) other useful reagents for amide bond formation, for example, as described by M. Bodansky, Principles of Peptide Synthesis, Springer-Verlag, 1984;
(vii) acylazides, e.g., wherein the azide group is generated from a preformed hydrazide derivative using sodium nitrite, as described by Wetz et al., Anal. Biochem. 58:347,1974; and
(viii) imidoesters, which form stable amidines on reaction with amino groups, for example, as described by Hunter and Ludwig, J. Am. Chem. Soc. 84:3491, 1962.
Aldehydes and ketones may be reacted with amines to form Schiff s bases, which may advantageously be stabilized through reductive amination.
Alkoxylamino moieties readily react with ketones and aldehydes to produce stable alkoxamines, for example, as described by Webb et al., in Bioconjugate Chem. 1:96, 1990.

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169, 1947. Carboxyl modifying reagents such as carbodiimides, which react through O-acylurea formation followed by amide bond formation, may also be employed.

It will be appreciated that functional groups in the targeting group may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

### Example 40

### Protection and deprotection of functional groups on L and T

The synthesis of wortmannin conjugates may involve the selective protection and deprotection of alcohols, amines, ketones, sulfhydryls or carboxyl functional groups of the linker, and/or the targeting group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kociensid, Protecting Groups, Georg Thieme Verlag, 1994.

### Example 41

### Exemplary Targeting Groups

Examples of targeting groups that can be used in the wortmannin conjugates of the invention are provided, without limitation, in Tables 5a and 5b. Exemplary commercially available peptides and their analogs are listed in Table 5a, followed by their respective BACHEM catalogue number.

TABLE 5a
Targeting Groups
Exemplary Targeting Groups Having Affinity for an In Vivo Site

| Targeting Group | Classification | Target |
|---|---|---|
| trastuzumab (Herceptin ™) | Antibody | Her2 |
| cetuximab (Erbitux ™) | Antibody | EGFR |
| bevacizumab (Avastin ™) | Antibody | VEGF |
| Abciximab (Reopro ™) | Antibody fragment | Platelet integrin IIb/IIIa (alpha IIb beta |
| | | 3, CD41/CD61)(ii) vitronectin receptor |
| Rituximab (Rituxan ™) | Antibody | CD20 |
| RGD peptides | Peptide | avB3 integrin |
| Bombesin-like peptides | Peptide | Gastrin releasing peptide receptor, see |
| | | Example 13. |
| | Bombesin (H-2155) | |
| | (Leu¹³-(®)-Leu¹⁴)-Bombesin (H-7075) | |
| | (Lys³)-Bombesin (H-2160) | |
| | (D-Phe¹²)-Bombesin (H-3038 | |
| | (D-Phe¹²,Leu¹⁴)-Bombesin (H-7070) | |
| | (Tyr⁴)-Bombesin (H-2165) | |
| | (Tyr⁴,D-Phe¹²)-Bombesin (H-9065) | |
| | (D-Cys⁶,Asn⁷,D-Ala¹¹,Cys¹⁴)-Bombesin (6-14) (H-8465) | |
| | (D-Phe⁶,Leu¹³-(®)-p-Chloro-Phe¹⁴)-Bombesin (6-14) (H-3028) | |
| | (D-Phe⁶,Leu-NHEt¹³,des-Met¹⁴)-Bombesin (6-14) (H-3042) | |
| | Bombesin (8-14) (H-2170) | |
| | Cyclo(-D-Phe-His-Trp-Ala-Val-Gly-His-Leu-Leu) (H-8470) | |
| Somatostatin-like peptides | Peptide | Somatostatin receptor |
| | Cortistatin-17 (H-5536) | |
| | 3-Mercaptopropionyl-Tyr-D-Trp-Lys-Val-Cys-p-chloro-D-Phe-NH₂ | |
| | (H-9505) | |
| | 3-Mercaptopropionyl-Tyr-D-Trp-Lys-Val-Cys-Phe-NH₂ (H-8460) | |
| | H-D-2-Nal-Cys-Tyr-D-Trp-Lys-Val-Cys-2-Nal-NH₂ (H-2126) | |
| | Octreotide (H-5972) | |
| | H-D-Phe-Cys-Tyr-D-Trp-Arg-Thr-Pen-Thr-NH₂ (H-3698) | |
| | H-D-Phe-Cys-Tyr-D-Trp-Orn-Thr-Pen-Thr-NH₂ (H-2186) | |
| | Cyclo-Somatostatin (H-2485) | |
| | Somatostatin-14 (H-1490) | |
| | Somatostatin-14 (reduced) (H-4662) | |
| | (Des-Ala¹,des-Gly²,His^{4,5},D-Trp⁸)-Somatostatin-14 (H-2495) | |
| | (D-Phe⁷)-Somatostatin-14 (H-4664) | |
| | (D-Ser¹³)-Somatostatin-14 (H-4666) | |
| | (D-Trp⁸)-Somatostatin-14 (H-3198) | |
| | (D-Trp⁸,D-Cys¹⁴)-Somatostatin-14 (H-1500) | |
| | Tyr-Somatostatin-14 (H-4995) | |
| | (Tyr¹)-Somatostatin-14 (H-5000) | |
| | (Tyr¹¹)-Somatostatin-14 (H-1495) | |
| | Somatostatin-14 (2-9) (H-4696) | |
| | Somatostatin-14 (3-10) (H-4702) | |
| | Somatostatin-14 (3-14) (H-4774) | |
| Somatostatin-like peptides | (D-Phe⁵,Cys^{6,11},N-Me-D-Trp⁸)-Somatostatin-14 (5-12) amide (H-5648) | |
| continued | Somatostatin-14 (7-14) (H-4698) | |
| | Somatostatin-25 (H-9580) | |
| | Somatostatin-28 (H-4955) | |
| | (Leu⁸,D-Trp²²,Tyr²⁵)-Somatostatin-28 (H-3202) | |
| | Tyr-Somatostatin-28 (H-4990) | |
| | Somatostatin-28 (1-12) (H-4945) | |
| | Somatostatin-28 (1-14) (H-4950) | |
| | (Tyr¹²)-Somatostatin-28 (1-14) (H-4960) | |
| EPPT1 peptide | Peptide | Muc-1 |
| | YCAREPPTRTFAYWG | See Moore et al., Can. Res. 64: 1821 |
| | | (2004). |
| Folate | Low MW molecule | Folate receptor & see example 14. |
| Methotrexate | Low MW molecule | Folate analogue |

Exemplary Anticancer Agents

| Class | Type of Agent | Nonproprietary Names | Cancers |
|---|---|---|---|
| Alkylating agents | Nitrogen mustards | mechlorethamine | Hodgkin's disease, |
| | | | non-Hodgkin's lymphomas |
| | | Cyclophosphamide | Acute and chronic |
| | | Ifosfamide | lymphocytic, leukemias, |
| | | | Hodgkin's disease, non- |
| | | | Hodgkin's lymphomas, |
| | | | multiple |
| | | | myeloma, neuroblastoma, |
| | | | breast, ovary, lung, Wilms' |
| | | | tumor, cervix, testis, soft-tissue |
| | | | sarcomas |
| | | Melphalan | Multiple myeloma, breast, |
| | | | ovary |
| | | Chlorambucil | Chronic lymphocytic leukemia, |
| | | | Primary macroglobulinemia, |
| | | | Hodgkin's disease, non- |
| | | | Hodgkin's lymphomas |
| | | Uracil mustard | Leukemia |
| | | Estramustine | Solid Tumors |
| | Ethylenimines and | Mitomycin C | Colorectal, ocular |
| | Methylmelamines | AZQ | Primary brain tumors |
| | | Thiotepa | Bladder, breast, ovary |
| | Alkyl Sulfonates | Busulfan | Chronic myelogenous leukemia |
| | | Hepsulfam | |
| | Nitrosoureas | Carmustine | Hodgkin's disease, non- |
| | | | Hodgkin's lymphomas, |
| | | | primary brain tumors, multiple |
| | | | myeloma, malignant melanoma |
| | | Lomustine | Hodgkin's disease, non- |
| | | | Hodgkin's lymphomas, |
| | | | primary brain tumors, small- |
| | | | cell lung |
| | | Sernustine | Primary brain tumors, stomach, |
| | | | colon |
| | | Streptozocin | Malignant pancreatic |
| | | | insulinama, malignant |
| | | | carcinoid |
| | Triazines | Dacarbazine | Malignant melanoma, |
| | | | Hodgkin's disease, soft-tissue |
| | | | sarcomas |
| | Platinum | Cisplatin | Testis, ovary, bladder, head and |
| | Complexes | Carboplatin | neck, lung, thyroid, cervix, |
| | | | endometrium, neuroblastoma, |
| | | | osteogenic sarcoma |
| | Methyl Hydrazine | Procarbazine | Hodgkin's discase |
| | Derivative | | |
| Antimetabolites | Folic Acid | Methotrexate | Acute lymphocytic leukemia, |
| | Antagonists | Trimetrexate | choriocarcinoma, mycosis |
| | | | fungoides, breast, head and |
| | | | neck, lung, osteogenic sarcoma |
| | Pyrimidine | Fluouracil | Breast, colon, stomach, |
| | Antagonists | Floxuridine | pancreas, ovary, head and neck, |
| | | | urinary bladder, skin, |
| | | | adenocarcinomas |
| | | Cytarabine | Acute myelogenous and acute |
| | | | lymphocytic leukemias |
| | | Fludarabine Phosphate | Lymphoproliferative disease |
| | | Capecitabine | Breast, renal cell, prostate |
| | | Azacitidine | acute leukemias |
| | Purine | Thioguanine | Acute myelogenous, acute |
| | Antagonists | | lymphocytic and chronic |
| | | | myelogenous leukemias |
| | | Mercaptopurine | Acute lymphocytic, acute |
| | | | myelogenous and chronic |
| | | | myelogenous leukemias |
| | | Allopurine | leukemias |
| | | Cladribine | Hairy cell leukemia |
| | | Gemcitabine | Pancreatic, soft tissue |
| | | | carcinomas |
| | | Pentostatin | Hairy cell leukemia, mycosis |
| | | | fungoides; chronic lymphocytic |
| | | | leukemia |
| Antimitotic | | Vinblastine | Hodgkin's disease, non- |
| Agents | | | Hodgkin's lymphomas, breast, |
| | | | testis |
| | | Vincristine | Acute lymphocytic leukemia, |
| | | | neuroblastoma, Wilms′ tumor, |
| | | | rhabdomyosarcoma, Hodgkin's |
| | | | disease, non-Hodgkin's |
| | | | lymphomas, small-cell lung |
| DNA Topoisomerase II Ihibitors | | Etoposide | Testis, small-cell lung, oat-cell |
| | | Teniposide | lung, breast, Hodgkin's disease, |
| | | | non-Hodgkin's lymphomas, |
| | | | acute myelogenous leukemia, |
| | | | Kaposi's sarcoma |
| DNA Topoisomerase I Ihibitors | | Topotecan | Ovarian, colorectal |
| | | Irinotecan | |
| | | Camptothecin | |
| | | 9-Aminocamptothecin | |
| Taxanes | | Paclitaxel | Breast |
| | | Docetaxel | |
| DNA Intercalators | | Daunorubicin | Acute myelogenous and acute |
| | | | lymphocytic leukemias |
| | | Doxorubicin | Ewing's sarcoma, |
| | | | osteosarcoma, |
| | | | rhabdomyosarcomas, |
| | | | Hodgkin's disease, non- |
| | | | Hodgkin's lymphomas, acute |
| | | | leukemias, multiple myeloma, |
| | | | breast, genitourinary, thyroid, |
| | | | lung, ovarian, endometrial, |
| | | | testicular, stomach, |
| | | | neuroblastoma |
| | | Dactinomycin | Choriocarcinoma, Wilms′ |
| | | | tumor, rhabdomyosarcoma, |
| | | | testis, Kaposi's sarcoma |
| | | Idarubincin | Acute myeloid leukemia |
| | | Plicamycin | Testicular cancer |
| | | Mitomycin | Squamous sell carcinomas, |
| | | | small bladder papillomas, |
| | | | adenocarcinomas, pancreas, |
| | | | lung, colon, stomach, cervix, |
| | | | breast, head and neck |
| | | Amsacrine | Acute myelogenous leukemia, |
| | | | ovarian cancer, lymphomas |
| | | Bleomycin | Testicular, head and neck, skin, |
| | | | esophagus, squamous cell, |
| | | | colorectal, lung, genitourinary |
| | | | tract, cervix, ovarian, breast, |
| | | | Hodgkin's disease, non- |
| | | | Hodgkin's lymphomas |
| Hormonal | Aromatase Inhibitors | Aminoglutethimide | Breast |
| Agents | | Anastrozole | |
| | 5-alpha-Reductase | Finasteride | Prostate |
| | Inhibitors | Ketoconazole | |
| | Estrogen and | Tamoxifen | Breast |
| | Androgen Inhibitors | Flutamide | Prostate |
| | Gonadotropin | Leuprolide | Prostate |
| | Releasing Hormone | Goserelin | |
| | Agonists | | |
| Tyrosine Kinase | ABL Inhibitors | Gleevec ™ (Novartis) | chronic myelogenous leukemia |
| Inhibitors | | | or acute lymphoblastic |
| | | | leukemia |
| | PDGFR Inhibitors | Leflunomide (Pharmacia), | gastrointestinal stromal tumor, |
| | | SU5416 (Pharmacia), | small cell lung cancer, |
| | | SU6668 (Pharmacia), | glioblastoma multiforme, and |
| | | PTK787 (Novartis) | prostate cancer |
| | EGFR Inhibitors | Iressa ™ (AstraZeneca), | non-small-cell lung cancer, |
| | | Tarceva ™, (Oncogene | breast cancer, ovarian cancer, |
| | | Science), | bladder cancer, prostate cancer, |
| | | trastuzumab (Genentech), | salivary gland cancer, |
| | | Erbitux ™ (ImClone), | pancreatic cancer, endometrial |
| | | PKI166 (Novartis), | cancer, colorectal cancer, |
| | | GW2016 | kidney cancer, head and neck |
| | | (GlaxoSmithKline), | cancer, glioblastoma |
| | | EKB-509 (Wyeth), | multiforme |
| | | EKB-569 (Wyeth), | |
| | | MDX-H210 (Medarex), | |
| | | 2C4 (Genentech), | |
| | | MDX-447 (Medarex), | |
| | | ABX-EGF (Abgenix), | |
| | | CI-1033 (Pfizer) | |
| | VEGFR Inhibitors | Avastin ™ (Genentech), | any solid tumor |
| | | IMC-1C11 (ImClone), | |
| | | ZD4190 (AstraZeneca), | |
| | | ZD6474 (AstraZeneca) | |
| | Trk Inhibitors | CEP-701 (Cephalon), | prostate cancer, pancreatic |
| | | CEP-751 (Cephalon) | cancer |
| | Flt-3 Inhibitors | MLN518 (Millenium), | acute myeloid leukemia |
| | | PKC412 (Novartis) | |
| | mTOR inhibitors | Everolimus, | Glioblastoma Multiforme, renal |
| | | Temsirolimus, | cell carcinoma |
| | | Sirolimus | |

| | | |
|---|---|---|
| Retinoic Acid Derivatives | 13-cis-retinoic acid, | Acute promyelocytic leukemia, |
| | isotretinoin, | head and neck squamous cell |
| | retinyl palmitate, | carcinoma |
| | 4-(hydroxycarbophenyl) | |
| | retinamide | |
| Hypoxia-Selective Cytoxins | Misonidazole | Head and neck |
| | Nitracrine | Breast |
| Miscellaneous | Mitoxantrone | Acute acute myelogenous |
| Agents | | leukemia non-Hodgkin's |
| | | lymphoma's, breast |
| | Hydroxyurea | Chronic myelogenous |
| | | leukemia, polycythemia vera, |
| | | essental thrombocytosis, |
| | | malignant melanoma |
| | L-Asparaginase | Acute lymphocytic leukemia |
| | Interferon alfa | Hairy cell leukemia., Kaposi's |
| | | sarcoma, melanoma, carcinoid, |
| | | renal cell, ovary, bladder, non- |
| | | Hodgkin's lymphomas, |
| | | mycosis fungoides, multiple |
| | | myeloma, chronic |
| | | myelogenous leukemia |
| | Mitotane | Adrenal carcinoma |

Exemplary Anti-Inflammatory Agents

Non Steroidal Anti-Inflammatory Drugs
naproxen
diclofenac
aspirin
sulindac
diflunisal
piroxicam
indomethacin
ibuprofen
nabumetone
5-amino salicylic acid
salicylsalicylic acid (salsalate)
fenoprofen
flurbiprofen
ketoprofen
meclofenamate
meloxicam
oxaprozin
sulindac
tolmetin
COX-2 Inhibitors
rofecoxib
celecoxib
valdecoxib
lumiracoxib
Anti-inflammatory Biologics
inflixamab
adelimumab
etanercept
CDP-870
rituximab
atlizumab
Corticosteroids
6α-fluoroprednisolone
6α-methylprednisolone
beclomethasone
betamethasone
budesonide
clobetasol
clobetasone
clocortolone
desonide
desoximethasone
dexamethasone
dichlorisone
diflorasone
diflucortolone
doxibetasol
fludrocortisone
flumethasone
fluocinonide
9-fluorocortisone
fluorohydroxyandrostenedione
fluorometholone
fluoxymesterone
flupredidene
fluprednisolone
flurandrenolide
halometasone
halopredone
hydrocortisone
6-hydroxydexamethasone
isoflupredone
isoprednidene
triamcinolone

TABLE 5b
Targeting Groups

| Ex- | | | |
|---|---|---|---|
| am- | | | Targeting Group |
| ple | WmC20 conjugate | Targeting Group | Function |
| 12 | 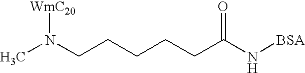 | Albumin (H₂N-BSA) | Increase Blood t1/2 |
| 13 | 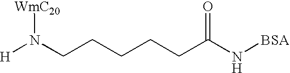 | Albumin (H₂N-BSA) | Increase Blood t1/2 |
| 14 | 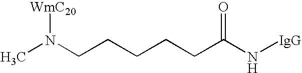 | Monoclonal or polyclonal Ab (H₂N-IgG) | Membrane Receptor |
| 15 | 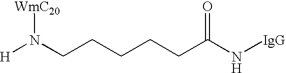 | Monoclonal or polyclonal Ab (H₂N-IgG) | Membrane Receptor |
| 16 | 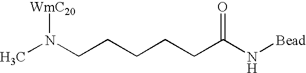 | Bead (H₂N-Bead) | Local Release |
| 17 | 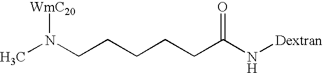 | Dextran (H₂N-Dextran) | Increase Blood t1/2 |
| 18 | 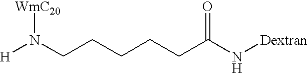 | Dextran (H₂N-Dextran) | Increase Blood t1/2 |
| 19 | WmC₂₀ -NH-Albumin | Albumin | Increase Blood t1/2 |
| 20 | 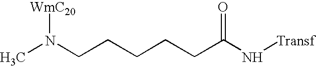 | Transferrin (H₂N-Transf) | Bind transferrin receptor |
| 21 | 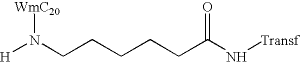 | (H₂N-Transf) | Bind transferrin receptor |
| 22 | 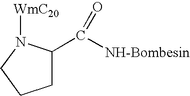 | Bombesin Peptide (H₂N-Bombesin) | GRP or NMB receptors |
| 23 | 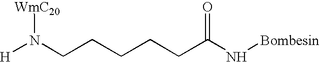 | Bombesin | GRP or NMB receptors |
| 24 | 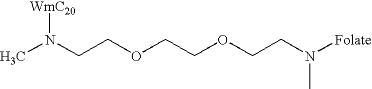 | Folate | Folate receptor |
| 25 | WmC₂₀-NH-cetiximab | cetiximab | EGFR receptors |
| 26 | WmC₂₀-NH-trastuzumab | trastuzumab | Her2 receptors |
| 27 | WmC₂₀-NH-abciximab | abciximab | GP IIb/IIa receptors |
| 28 | 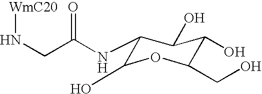 | 2-amino glucose | Physical Property, hydrophilicity |
| 29 | 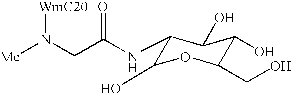 | 2-amino glucose | Physical Property, hydrophilicity |
| 30 | 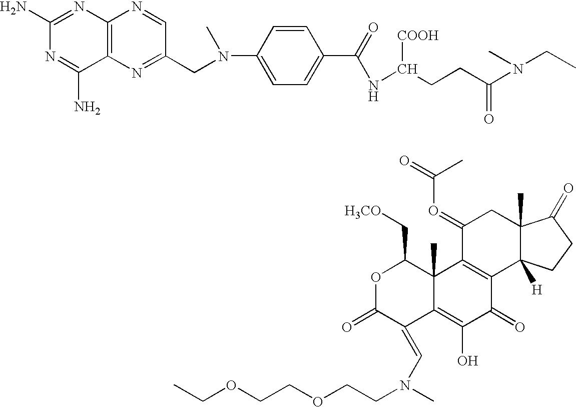 | Methotrexate | Enhanced Potency |
| 31 | 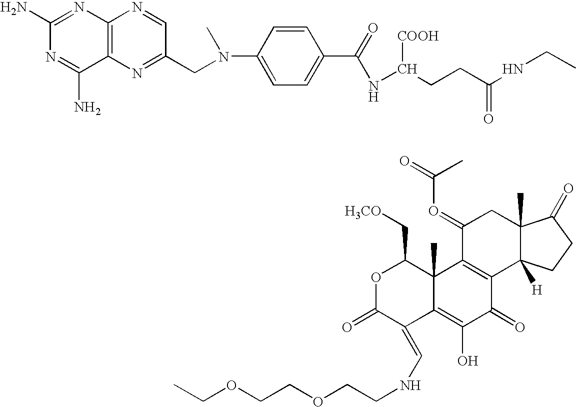 | Methotrexate | Enhanced Potency |
| 32 | 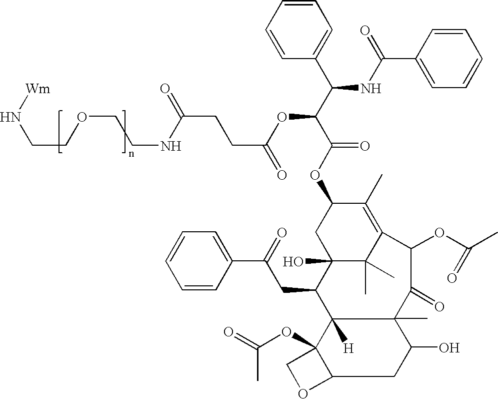 | Paclitaxel | Enhanced Potency |
| 33 | 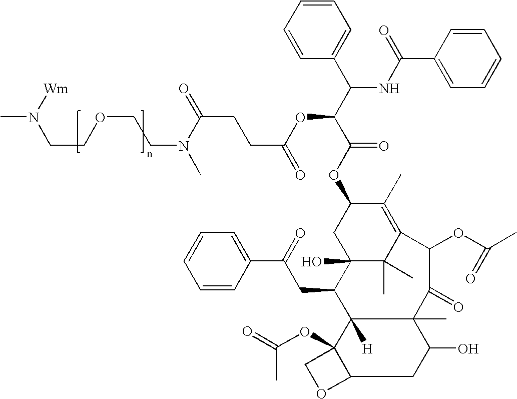 | Paclitaxel | Enhanced Potency |

### Example 42

### Exemplary WmC20 derivatives (compound of formula W−L)

Examples of WmC20 derivatives that can be used to make WmC20 conjugates of the invention are, without limitation, provided in Tables 6a and 6b.

TABLE 6a
WmC20 Derivatives

| Example | Wortmannin Derivative | Comment |
|---|---|---|
| 1 | 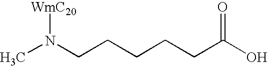 | W-L useful for the attachment of amine bearing targeting groups (T). Compound 2a of FIG. 4. |
| 2 | 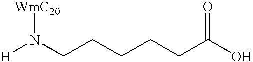 | W-L useful for the attachment of amine bearing targeting groups (T). Compound 2b of FIG. 4. |
| 3 | 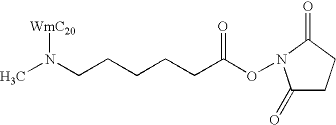 | Activated W-L. Compound 3a of FIG. 4. |
| 4 | 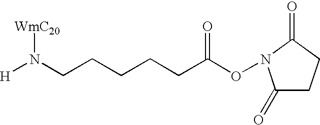 | Activated W-L. Compound 3b of FIG. 4. |
| 5 | 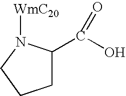 | W-L useful for the attachment of amine bearing targeting groups (T). |
| 6 | 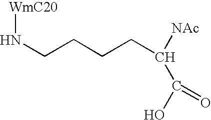 | W-L useful for the attachment of amine bearing targeting groups (T). |
| 7 | 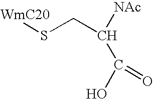 | W-L useful for the attachment of amine bearing targeting groups (T). |
| 8 | 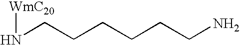 | W-L useful for the attachment of carboxyl bearing targeting groups (T). |
| 9 | 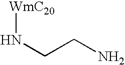 | W-L useful for the attachment of carboxyl bearing targeting groups (T). |
| 10 | 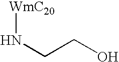 | W-L useful for the attachment of carboxyl bearing targeting groups (T). |
| 11 | 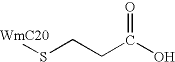 | W-L useful for the attachment of amine bearing targeting groups (T). |

TABLE 6b
Linkers for use in WmC20 derivatives of formula W-L

| | W-reactive | T-reactive | Conjugation | |
|---|---|---|---|---|
| Linker | Group | Group | Chemistry | Ref. |
| 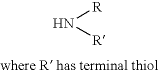 | 1° or 2° amine | —SH | SIA, SPDP | |
| 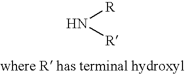 | 1° or 2° amine | —OH, | Thionyl chloride | 1 |
| 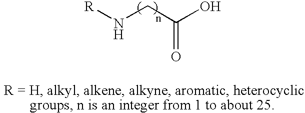 | 1° or 2° amine | —COOH | CDI, CDI & NHS, acid chlorides | 1 |
| 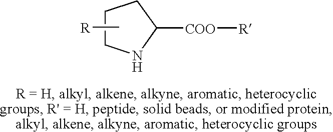 | 2° amine | —COOH | CDI, NHSCDI | 1 |
| R′—NH—CHR—COOH^{a} | 1° or 2° | COOH | CDI, NHS | 1 |
| R is amino acid side chain of natural or unnatural amino | amine | | | |
| acid | | | | |
| R′—NH—XXX—COOH^{a} | 1° or 2° | COOH | CDI, NHS | 1 |
| | amine | | | |
| R′—NH—PEG—COOH^{a} | 1° or 2° | COOH | CDI, NHS | |
| PEG = —(CH₂CH₂O)n— | amine | | | |
| HS-Y-R′ | Thiol | COOH | CDI, NHS or | 1-3 |
| Y = natural and unnatural amino acids, peptide, protein, | | and | other coupling | |
| alkyl, alkene, alkyne, aromatic, heterocyclic groups | | other | agents | |
| R′ = COOH, NH₂, OH, or other functions | | functions | | |
| (O—CO—CH₂CH₂—S)— | Thiol | COOH | CDI, NHS | 4 |
| —R—Y—CX—Z—N═ | | hydrazone | | 5, 6 |
| R = H, alkyl, alkene, alkyne, aromatic, heterocyclic | | | | |
| groups, —(CH₂CH₂CONH)n— Y = NH, S, O, X═ | | | | |
| ═NH, ═S, ═O; Z = NH, or aromatic amino groups | | | | |

^{a}R′ = H, C₁₋₁₀ alkyl, C₁₋₁₀ heteroalkyl, C₂₋₁₀ alkene, C₂₋₁₀ alkyne, a C₅₋₁₀ aryl, a cyclic system of 3 to 10 atoms.
References for Table 6a
1. Wipf et al., Organic & biomolecular chemistry 2:1911 (2004).
2. Ihle et al., Mol. Cancer Ther. 3:763 (2004).
3. Norman et al., J. Med. Chem. 39:1106 (1996).
4. Oishi et al., J. Am. Chem. Soc. 127:1624 (2005).
5. Lau et al., Bioorg. Med. Chem. 3:1305 (1995).
6. Kaneko et al., Bioconjug. Chem. 2:133 (1991).

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A compound of formula I:
W−L−T (I)
wherein
W is a wortmannin C20 derivative;
T is a non-naturally occurring targeting group; and
L is a linker which forms a covalent bond with said wortmannin C20 derivative at position C20 and forms a covalent bond with said targeting group, and wherein said compound comprises a thioether or amine substituent at position C20 of said wortmannin C20 derivative.

2. A compound of formula I:
W−L−T (I)
wherein said compound is substantially pure;
W is a wortmannin C20 derivative;
T is a targeting group; and
L is a linker which forms a covalent bond with said wortmannin C20 derivative at position C20 and forms a covalent bond with said targeting group, and wherein said compound comprises a thioether or amine substituent at position C20 of said wortmannin C20 derivative.

3. The compound of claims 1 or 2, wherein said linker is described by formula IIa:
G²-(X¹)—(R₁₀)-(Z²)ₛ-(Y¹)ᵤ-(Z¹)ₒ-G¹ (IIa)
wherein
G¹ is a bond between said linker and said targeting group;
G² is a bond between said wortmannin C20 derivative and said linker,
X¹ is S or NR₁₁;
each of Z¹ and Z² is, independently, selected from O, S, and NR₁₂;
R₁₂ is selected from hydrogen, C₁₋₁₀ alkyl, C₁₋₁₀ heteroalkyl, C₂₋₁₀ alkene, C₂₋₁₀ alkyne, and C₅₋₁₀ aryl;
Y¹ is selected from carbonyl, thiocarbonyl, sulphonyl, or phosphoryl;
each of o, s, and u is, independently, 0 or 1; and
R₁₁ is selected from hydrogen, C₁₋₁₀ alkyl, C₂₋₁₀ alkene, C₂₋₁₀ alkyne, and C₅₋₁₀ arylgroup; and
R₁₀ is a C₁₋₁₀ alkyl, C₁₋₁₀ heteroalkyl, C₂₋₁₀ alkene, C₂₋₁₀ alkyne, a C₅₋₁₀ aryl, a cyclic system of 3 to 10 atoms, —(CH₂CH₂O)_{q}CH₂CH₂— in which q is an integer of 1 to 8; or
R₁₀ and R₁₁ combine to form a cyclic system of 3 to 10 atoms.

4. The compound of claim 3, wherein the linker is described by the formula IIb:
G²-(NR₁₃)—R₁₄—C(O)-G¹ (IIb)
wherein
G¹ is a bond between said linker and said targeting group;
G² is a bond between said wortmannin C20 derivative and said linker,
R₁₃ is selected from hydrogen, C₁₋₁₀ allyl, C₁₋₁₀ heteroalkyl, C₂₋₁₀ alkene, C₂₋₁₀ alkyne, and C₅₋₁₀ arylgroup; and
R₁₄ is a C₁₋₁₀ alkyl, C₁₋₁₀ heteroalkyl, C₂₋₁₀ alkene, a C₂₋₁₀ alkyne, a C₅₋₁₀ aryl, a cyclic system of 3 to 10 atoms, or —(CH₂CH₂O)_{q}CH₂CH₂— in which q is an integer of 1 to 8; or
R₁₃ and R₁₄ combine to form a cyclic system of 3 to 10 atoms.

5. The compound of claims 1 or 2, said compound is formed by reaction of thiol group or amino group of said targeting group T with a wortmannin-like compound or a wortmannin C20 derivative.

6. The compound of claims 1 or 2, wherein said wortmannin-like compound is selected from viridin, viridiol, demethoxyviridin, demethoxyviridiol, wortmannin, wortmannolone, 17-hydroxywortmannin, 11-desacetoxywortmannin, and Δ9,11 dehydrodesacetoxywortmannin.

7. The compound of claims 1 or 2, wherein said targeting group is a peptide, peptidomimetic, low molecular weight ligand, protein, polymer, solid support, anticancer agent, or anti-inflammatory agent.

8. The compound of claim 7, wherein said targeting group is a polymer comprising a polypeptide, polysaccharide, or polyethyleneglycol.

9. The compound of claim 7, wherein said targeting group is a protein.

10. The compound of claim 9, wherein said protein is an antibody or fragment thereof.

11. The compound of claim 10, wherein said antibody or fragment thereof is selected from rituximab, cetuximab, trastuzumab, bevacizumab, and abciximab.

12. The compound of claim 7, wherein said targeting group is a peptide or peptidomimetic selected from bombesin-like peptides, somatostatin-like peptide, RGD peptides, and EPPT1 peptide.

13. The compound of claim 7, wherein said targeting group is a low molecular weight ligand selected from methotrexate, trimetrexate, and folate.

14. The compound of claim 7, wherein said targeting group is a solid support.

15. The compound of claim 7, wherein said targeting group is an anticancer agent selected from alkylating agents, folic acid antagonists, pyrimidine antagonists, purine antagonists, antimitotic agents, DNA topomerase II inhibitors, DNA topomerase I inhibitors, taxanes, DNA intercalators, aromatase inhibitors, 5-alpha-reductase inhibitors, estrogen inhibitors, androgen inhibitors, gonadotropin releasing hormone agonists, retinoic acid derivatives, and hypoxia selective cytotoxins.

16. The compound of claim 7, wherein said targeting group is an anti-inflammatory agent selected from non steroidal anti-inflammatory drugs, COX-2 inhibitors, anti-inflammatory biologics, and corticosteroids.

17. The compound of claims 1 or 2, wherein said compound is further described by any of formulas IIIa to IIIi: 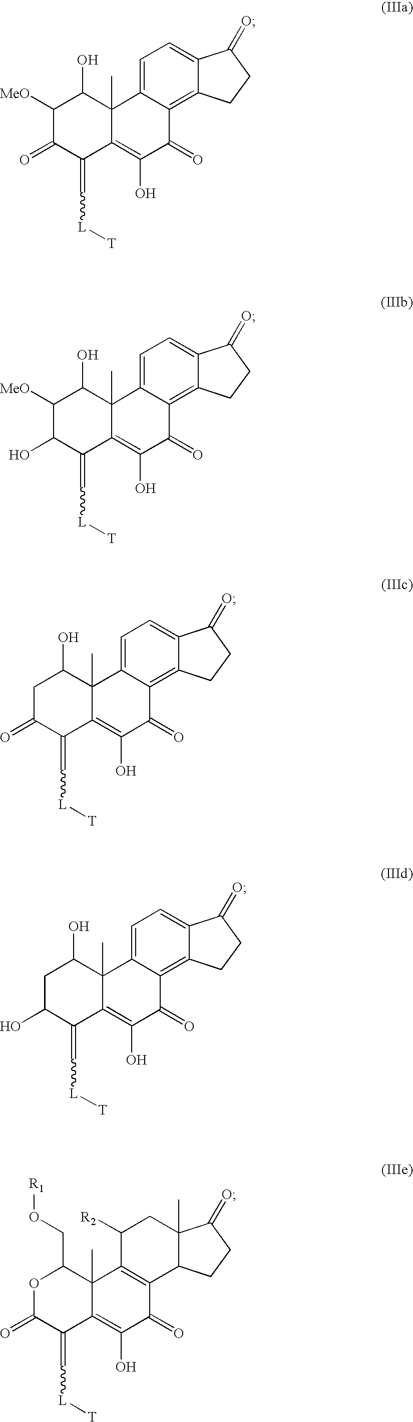 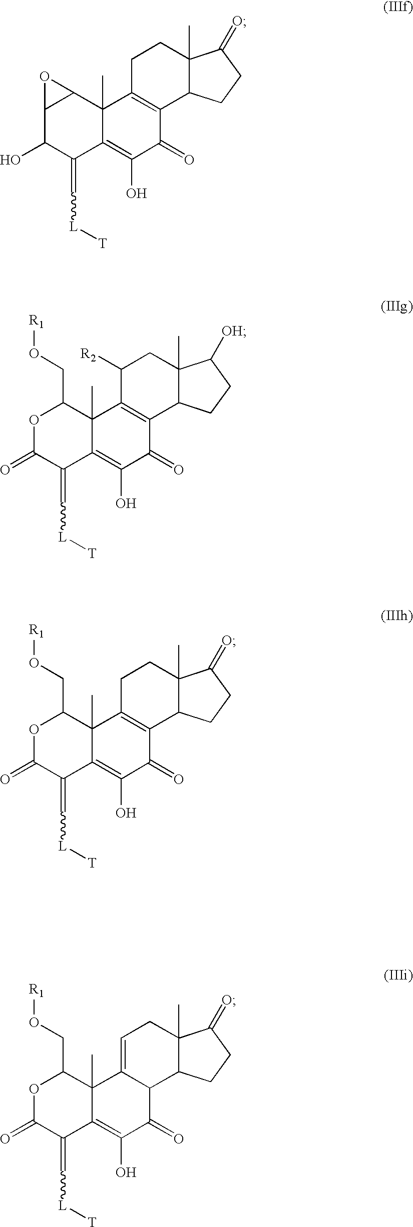
wherein
T is a targeting group;
L is a linker;
R₂ is OH, OR₃, or OC(O)R₃; and
each of R₁ and R₃ is, independently, selected from C₁₋₁₀ alkyl, C₁₋₁₀ heteroalkyl, C₂₋₁₀ alkene, and C₂₋₁₀ alkyne.

18. The compound of claim 17, wherein said compound is described by formulas IIIe, IIIg, IIIh, or IIIi, and wherein R₁ and R₃ are methyl.

19. The compound of claim 17, wherein said linker L is a bond linking said wortmannin C20 derivative to said targeting group.

20. The compound of claim 19, wherein said compound is formed is formed via a ring-opening attack of the C20 position of a wortrmannin-like compound by a primary amine, secondary amine, or thiol present on said targeting group.

21. The compound of claim 17, wherein L is a bond linking said wortmannin C20 derivative to said targeting group.

22. An article comprising a composition of formula I:
W−L−T (I)
wherein
W is a wortmannin C20 derivative;
T is a solid support on or within said article;
L is a linker which forms a covalent bond with said wortmannin C20 derivative at position C20 and forms a covalent bond with said solid support;
and wherein said composition comprises a thioether or amine substituent at position C20 of said wortmannin C20 derivative.

23. The article of claim 22, wherein said article is an implantable medical device.

24. The article of claim 23, wherein said article is a stent or a drug delivery device.

25. The article of claim 22, wherein said article further comprises a radioactive isotope.

26. A pharmaceutical composition comprising a compound of any of claims 1-21 in any pharmaceutically acceptable form and a pharmaceutically acceptable carrier or diluent.

27. A method for reducing PI3 kinase activity in a cell, said method comprising contacting said cell with a compound of any of claims 1-21 in an amount sufficient to reduce said activity.

28. A method for treating an inflammatory condition in a mammal, said method comprising administering to said mammal a compound of any of claims 1-21 in an amount sufficient to treat said condition.

29. A method for treating a proliferative disorder in a mammal, said method comprising administering to said mammal a compound of any of claims 1-21 in an amount sufficient to treat said disorder.

30. A method for treating a Candida albicans infection in a mammal, said method comprising administering to said mammal a compound of any of claims 1-21 in an amount sufficient to treat said infection.

31. A process for the preparation of a compound of formula IV, said process comprising the step of reacting a compound of formula V with a targeting group bearing a primary or secondary amine, 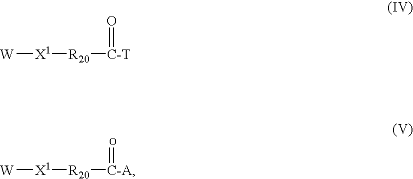
wherein
W is a wortmannin C20 derivative;
T is a targeting group bearing a primary or secondary amine;
A is N-hydroxysuccinimidyl ester or N-hydroxysulfosuccinimidyl ester;
X¹ is S or NR₂₁;
R₂₁ is selected from hydrogen, C₁₋₁₀ alkyl, C₁₋₁₀ heteroalkyl, C₂₋₁₀ alkene, C₂₋₁₀ alkyne, and C₅₋₁₀ aryl; and
R₂₀ is selected from C₁₋₁₀ alkyl, C₁₋₁₀ heteroalkyl, a C₂₋₁₀ alkene, a C₂₋₁₀ alkyne, a C₅₋₁₀ aryl, a cyclic system of 3 to 10 atoms, and —(CH₂CH₂O)_{q}CH₂CH₂— in which q is an integer of 1 to 8; or
R₂₀ and R₂, combine to form a cyclic system of 3 to 10 atoms.

32. A process for the preparation of a compound of claim 1, said process comprising reacting a compound of formula VI with a targeting group bearing a primary amine,
W—(X¹)—R₃₀ (VI)
wherein
W is a wortmannin C20 derivative;
X¹ is S or NR₃;
each of R₃₀ and R₃₁ is, independently, selected from C₁₋₁₀ alkyl, C₂₋₁₀ alkene, C₂₋₁₀ alkyne, C₅₋₁₀ arylgroup, and a cyclic system of 3 to 10 atoms, or R₃₀ and R₃₁ combine to form a cyclic system of 3 to 10 atoms.

33. The process of claim 32 wherein said targeting group is attached to a linker bearing a primary amino group.

34. The process of claim 32, wherein said targeting group bears an amino group and reacts directly with said compound of formula VI.

35. A compound of formula VII: 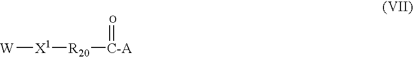
wherein
W is a wortrmannin C20 derivative;
A is N-hydroxysuccinimidyl ester or N-hydroxysulfosuccinimidyl ester;
X¹ is S or NR₂₁;
R₂₁ is selected from hydrogen, C₁₋₁₀ alkyl, C₁₋₁₀ heteroalkyl, C₂₋₁₀ alkene, C₂₋₁₀ alkyne, and C₅₋₁₀ aryl; and
R₂₀ is a C₁₋₁₀ alkyl, C₁₋₁₀ heteroalkyl, a C₂₋₁₀ alkene, a C₂₋₁₀ alkyne, a C₅₋₁₀ aryl, a cyclic system of 3 to 10 atoms, or —(CH₂CH₂O)_{q}CH₂CH₂— in which q is an integer of 1 to 8; or
R₂₀ and R₂₁ combine to form a cyclic system of 3 to 10 atoms.

36. The compound of claim 35, wherein X¹ is S.

37. The compound of claim 35, wherein X¹ is NR₂₁.

38. The compound of claim 37, wherein said compound is the N-hydroxysuccinimide ester of wortmannin C20-N(Me)-hexanoic acid or the N-hydroxysuccinimide ester of wortmannin C20-NH-hexanoic acid.

39. A compound of formula VIII:
W—N(CH₃)—[CH₂]_{n—COOH} (VI)
or a salt thereof, wherein,
W is a wortmannin C20 derivative; and
n is an integer of 2 to 10.
